(19) United States
(12) Patent Application Publication
(10) Pub. No.: US 2026/0071280 A1
(43) Pub. Date: Mar. 12, 2026

(54) METHODS TO PREDICT RESPONSE TO ICBT OF A PATIENT HAVING CANCER

(71) Applicant: Novigenix SA, Epalinges (CH)

(72) Inventors: Victoria WOSIKA, Epalinges (CH); Noushin HADADI, Epalinges (CH); Eric DURANDAU, Epalinges (CH); Laura CIARLONI, Epalinges (CH); Sahar Hosseinian EHRENSBERGER, Epalinges (CH); Sylvain MONNIER-BENOIT, Epalinges (CH)

(21) Appl. No.: 19/107,647

(22) PCT Filed: Sep. 1, 2023
(86) PCT No.: PCT/EP2023/074064

§ 371 Date: Feb. 28, 2025

(30) Foreign Application Priority Data
Sep. 7, 2022 (EP) 22194431.7

(51) Int. Cl. C12Q 1/6886; G16B 25/10; G16B 40/20; G16H 50/20

(52) U.S. Cl. CPC C12Q 1/6886; G16B 25/10; G16B 40/20; G16H 50/20; C12Q 2600/106; C12Q 2600/158

(57) ABSTRACT

The present invention relates to methods for determining or predicting if a patient having a predetermined disease, for example cancer, in particular metastatic melanoma, is responsive, or will respond to a treatment based on immune checkpoint inhibitor. The present invention also relates to computer-implemented methods for implementing said methods and to kits.

### FIELD OF THE INVENTION

The present invention relates to methods for determining or predicting if a patient having a predetermined disease, for example cancer, in particular metastatic melanoma, is responsive, or will respond to a treatment based on immune checkpoint inhibitor. The present invention also relates to computer-implemented methods for implementing said methods and to kits.

### BACKGROUND OF THE INVENTION

While immune checkpoint blockade therapy (ICBT) has revolutionized treatment of metastatic melanoma (MM) patients, still 40-60% of patients do not achieve a clinical benefit (1). Application of biomarkers before the start of treatment would limit the use of ICBT in patients that do not benefit from it, thereby preventing immune-related toxicity and enabling the rapid introduction of other, potentially more effective therapies.

Moreover, response biomarkers applied early during treatment may identify patients who need treatment extension without waiting the first radiological response evaluation at 12 weeks and avoid thus therapy discontinuation. Also, radiological response evaluation is sometimes equivocal.

Tissue-based predictive biomarkers, such as PDL1 expression or tumor mutational burden, are not validated in MM (1-2). Thus, non-invasive liquid biomarkers can be an attractive alternative.

Therefore, biomarkers that can both predict clinical outcome and help determining a patient's responsiveness to ICBT, for instance patient treated with immune checkpoint blockade therapy (e.g. anti-PD-1 and/or anti-CTLA4), are urgently needed.

### SUMMARY OF THE INVENTION

The present invention provides a method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising at least one gene selected among:
the Ribosomal biogenesis genes cluster,
the TCR signaling genes cluster,
the Cilia genes cluster,
the Interferon pathway genes cluster, or a combination of one or more thereof,
   wherein differential transcription and/or expression and/or activity level of the gene panel, in the biological sample, relative to the level of corresponding transcription and/or expression and/or activity level of the gene panel determined previously, is predictive of the patient's response to said treatment.

The present invention also provides a method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising at least one gene selected among:
the Cell Cycle genes cluster,
the Jak/Stat Signaling pathway genes cluster, and
the T cell/Immune tolerance genes cluster, or a combination of one or more thereof,
   wherein differential transcription and/or expression and/or activity level of the gene panel, in the biological sample, relative to the level of corresponding transcription and/or expression and/or activity level of the gene panel determined previously in a reference sample, is predicting that the patient is responsive to said treatment.

Also provided is a computer-implemented method for implementing a method for predicting if a patient having a predetermined disease will respond to a treatment based on immune checkpoint blockade therapy or treatment (ICBT) of the invention, said computer-implemented method comprising
i) scoring the level of transcription and/or expression and/or activity of a gene panel in the biological sample of the patient,
ii) comparing the determined score to the score of the gene panel determined previously, whereby difference in the score, in the biological sample, relative to the score of the gene panel determined previously, is predictive of the patient's response to said treatment.

Also provided is a computer-implemented method for implementing a method for determining if a patient having a predetermined disease is responsive to a treatment based on immune checkpoint blockade therapy or treatment (ICBT) of the invention, said computer-implemented method comprising
i) scoring the level of transcription and/or expression and/or activity of a gene panel in the biological sample of the patient,
ii) comparing the determined score of the gene panel determined previously, whereby difference in the score, in the biological sample, relative to the score of the gene panel determined previously, is indicative of whether the patient is responsive or not to said treatment.

Also provided is the use of a gene panel comprising at least one gene selected among
the Cell Cycle genes cluster,
the Jak/Stat Signaling pathway genes cluster, and
the T cell/Immune tolerance genes cluster,
or a combination of one or more thereof, for predicting if a patient having a predetermined disease will respond to a treatment based on immune checkpoint blockade therapy or treatment (ICBT).

Also provided is the of a gene panel comprising at least one gene selected among
the Cell Cycle genes cluster,
the Jak/Stat Signaling pathway genes cluster, and
the T cell/Immune tolerance genes cluster,
or a combination of one or more thereof, for determining if a patient having a predetermined disease is responsive to a treatment based on immune checkpoint blockade therapy or treatment (ICBT).

Also provided is a kit for performing a method according to the invention, said kit comprising
a) means and/or reagents for determining the level of transcription and/or expression and/or activity of said gene panel in a biological sample from said patient, and
b) instructions for use.

Also provided is a method of treatment of cancer, comprising
i) detecting in a biological sample obtained from said patient the level of transcription and/or expression and/or activity of a gene panel of the invention, and
ii) treating the patient based upon whether a differential transcription and/or expression and/or activity level of said gene panel, in the biological sample, relative to the level of corresponding transcription and/or expression and/or activity level of the gene panel determined previously, is predictive of the patient's response to said treatment.

Further provided is a method of treatment of cancer, comprising
i) detecting in a biological sample obtained from said patient the level of transcription and/or expression and/or activity of a gene panel of any one of the invention, and
ii) treating the patient based upon whether a differential transcription and/or expression and/or activity level of said gene panel, in the biological sample, relative to the level of corresponding transcription and/or expression and/or activity level of the gene panel determined previously, is determining that the patient is responsive or not to said treatment.

### DESCRIPTION OF THE FIGURES

FIG. 1: Gene expression difference in CB+ (Resp.) and CB− (Non Resp.) patients at baseline relative to a 7-gene signature of the TCR signaling cluster (A) and 6-gene signature from the Ribosome biogenesis cluster (B).

FIG. 2: ROC curve depicting the performance of the classifiers “119 genes” (A) and “best 25 genes” (B) for predicting response to therapy at baseline. AUC is indicated.

FIG. 3: Kaplan-Meier curves. Progression-free survival in patients classified as responder and non-responder by the 119-gene model (A), and the 25-gene model (B). Time is expressed in months.

FIG. 4: Gene expression difference in CB+ (Resp.) patients at baseline and 6 weeks relative to a 15-gene signature from the Cell Cycle cluster (A), 10-gene signature from the T cell/Immune tolerance cluster (B), and 4-gene signature from the JAK/STAT signalling cluster.

FIG. 5: ROC curve depicting the performance of the classifiers “141 genes” (A) and the “best 25 genes” (B) for identifying response to anti PD1/CTLA4 therapy at 6 weeks of treatment. AUC is indicated.

FIG. 6: Kaplan-Meier curves. Progression-free survival in patients classified as responder and non-responder by the 141-gene model. Time is expressed in months.

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms “comprise(s)” and “comprising” also encompass the more restricted ones “consist(s)”, “consisting” as well as “consist/consisting essentially of”, respectively.

As used in the specification and claims, the singular form “a”, “an” and “the” include plural references unless the context clearly dictates otherwise.

As used herein, “at least one” means “one or more”, “two or more”, “three or more”, etc. For example, at least one gene means one or more, two or more, three or more, four or more, etc . . . .

The term “about”, particularly in reference to a given quantity, number or percentage, is meant to encompass deviations of plus or minus ten percent (±10%). For example, about 5% encompasses any value between 4.5% to 5.5%, such as 4.5, 4.6, 4.7, 4.8, 4.9, 5, 4.1, 5.2, 5.3, 5.4, or 5.5.

As used herein the terms “subject”/“patient”, are well-recognized in the art, and are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease or disorder. However, in other aspects, the subject can be a normal subject. The term does not denote a particular age or sex. Thus, adult and newborn subjects, whether male or female, are intended to be covered. Preferably, the subject is a human, most preferably a human patient having a predetermined disease, more preferably the predetermined disease is a cancer.

In one aspect, the predetermined disease is a cancer, whether solid or liquid, selected from the non-limiting group comprising urothelial cancer, urinary bladder cancer, lung cancer, breast cancer, ovarian cancer, cervical cancer, uterus cancer, head and neck cancer, glioblastoma, hepatocellular carcinoma, colon cancer, rectal cancer, kidney cancer, prostate cancer, gastric cancer, bronchus cancer, pancreatic cancer, hepatic cancer, melanoma, brain cancer and skin cancer, or a combination of one or more thereof.

Preferably, the cancer is a melanoma, more preferably metastatic melanoma (MN).

In one aspect, the metastatic melanoma is a melanoma bearing a BRAF gene mutation (e.g. BRAF V600e gene mutation).

In one aspect, the treatment of the invention is based on immune checkpoint blockade therapy or treatment (ICBT). Preferably, said treatment based on ICBT is selected among the group comprising a PD-1 inhibitor, a PD-L1 inhibitor, a LAG-3 inhibitor, a TIM-3 inhibitor, a TIGIT inhibitor, a BTLA inhibitor and a CTLA-4 inhibitor, or combination of one or more thereof (e.g. CTLA-4 and PD-1 inhibitors, or LAG-3 and PD-1 inhibitors).

In a preferred aspect, the treatment based on ICBT comprises treatment with monoclonal antibodies (mAbs) specific to, or designed to bind with, PD-1, PD-L1, LAG-3, TIM-3, TIGIT, BTLA or CTLA-4, or a combination of one or more thereof (see e.g. Rotte, A. et al., 2019; Twomey, J. D. and Zhang, B. 2021).

Non-limiting examples of mAbs specific to PD-1 comprise nivolumab, pembrolizumab, dostarlimab, retifanlimab and cemiplimab.

Non-limiting examples of mAbs specific to PDL-1 comprise atezolizumab, avelumab, and durvalumab.

Non-limiting examples of mAbs specific to LAG-3 comprise favezelimab andrelatlimab.

Non-limiting examples of mAbs specific to TIM-3 comprise cobolimab, LY3321367, or sabatolimab.

Non-limiting examples of mAbs specific to TIGIT comprise tiragolumab and EOS-448 (devolpped by GSK and iTeos Therapeutics).

Non-limiting examples of mAbs specific to BTLA comprise IND (junishi biosciences) and talquetamab-tgvs.

Non-limiting examples of mAb specific to CTLA-4 consist of ipilimumab and tremelimumab.

As defined herein, patients were classified as responder (CB+) or non-responder (CB−). Patients were considered to have clinical benefit (responder, CB+) if they had a clinical progression-free survival (PFS) exhibiting both complete and/or partial response of at least 6 months. Conversely, patients with PFS lower than six months, showing stable disease or progression in disease were classified as having no clinical benefit (non-responder, CB−).

As discussed herein, the level of transcription and/or expression and/or activity of a gene panel may be expressed as a score. The score may be calculated as the mean, or the median, or the ratio or the sum, or the weighted mean, median or the sum, the ratio of the expression levels of the genes composing the panel in control samples (e.g. reference samples, baseline, . . . ) and disease samples.

Alternatively, the score may be calculated as the first component or multiple components of Principal Component Analysis (PCA), or Neural Network dimensional embeddings or any Dimensionality reduction method.

Also, it can be calculated as a probability of a prediction model using generalized linear models, or Lasso and Elastic-Net Regularized Generalized Linear Models, Sparse partial least squares regression, or nearest-centroid classification, or nearest shrunken centroid, or neural networks or random forest, or support vector machine, or naïve bayes, or K-means.

A “pre-defined score” refers to a mathematical formula that has been determined by fitting a predictive model at training phase on the training data set for instance by logistic regression. The fitted model will be used to calculate the score or predict the likelihood of being responsive to the therapy for each new patient. The bootstrap method or the cross-validation method with a ROC analysis can estimate the performances of the fitted model in each mathematical method. The present description provides an example of a model based on training.

Various techniques for determining differential transcription and/or expression and/or activity of a gene panel are known in the art.

For example, one may calculate differential expression of one gene in a test sample by, e.g. calculating the ratio (fold change) between the expression level of the gene in the test sample and the expression level of the gene in the reference sample or group of samples, or reference value.

Expression level can be measured as transcripts per million (TPM) by RNA seq, as Threshold cycles (Ct) by PCR, as probe fluorescence intensity by microarray, etc . . . .

Determining transcriptional changes in a group of samples for all the transcriptome is usually done using computational methods to determine differential gene expression in a full RNAseq dataset (e.g. 15000 genes). Different commonly used methods are, e.g., selected among the following software packages (open source): edgeR, DESeq2, limma, Cuffdiff, PoissonSeq, baySeq, etc . . . . Preferably, DESeq2 is used (Love, M. I. et al., 2014).

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker in the absence of treatment or after starting the treatment. These values or ranges can be obtained from a single patient or alternatively from a group of patients.

The transcripts of the genes of the invention can be detected and, alternatively, quantitated by a variety of methods including, but not limited to, microarray analysis, polymerase chain reaction (PCR), reverse transcriptase polymerase chain reaction (RT-PCR), Northern blot, serial analysis of gene expression (SAGE), immunoassay, mass spectrometry, and any RNA sequencing-based methods known in the art (such as e.g. whole transcriptome RNA seq, targeted RNA seq, single cell RNA seq, total RNA sequencing, mRNA sequencing, whole transcript RNA sequencing, 3′ RNA sequencing, long RNA sequencing, direct RNA sequencing).

It is understood that the expression level of the genes (e.g. biomarkers) in a sample can be determined by any suitable method known in the art. Measurement of the level of a gene can be direct or indirect. For example, the abundance levels of RNAs can be directly quantitated. Alternatively, the amount of a gene (biomarker) can be determined indirectly by measuring abundance levels of cDNAs, amplified RNAs or DNAs, or by measuring quantities or activities of RNAs, or other molecules that are indicative of the expression level of the gene (such as, e.g proteins). Preferably, the amount of a gene (biomarker) is determined indirectly by measuring abundance levels of cDNAs.

Biomarker which may be measured by microarray or RNA sequencing analysis can be expressed RNAs or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one aspect, the target polynucleotide molecules comprise RNA, including, but by no means limited to, total cellular RNA, poly(A)+ messenger RNA (mRNA) or a fraction thereof, cytoplasmic mRNA, or RNA transcribed from cDNA (i.e., cRNA; see, e.g., U.S. Pat. Nos. 5,545,522, 5,891,636, or 5,716,785). Methods for preparing total and poly(A)+RNA are well known in the art, and are described generally, e.g., in Sambrook, et al., Molecular Cloning: A Laboratory Manual (3rd Edition, 2001) as well as e.g. in Hong, M., Tao, S., Zhang, L. et al. RNA sequencing: new technologies and applications in cancer research. J Hematol Oncol 13, 166 (2020). RNA can be extracted from a cell of interest using guanidinium thiocyanate lysis followed by CsCl centrifugation, a silica gel-based column (e.g., RNeasy (Qiagen, Valencia, Calif) or StrataPrep (Stratagene, La Jolla, Calif.)), or using phenol and chloroform, as known in the art. Poly(A)+RNA can be selected, e.g., by selection with oligo-dT cellulose or, alternatively, by oligo-dT primed reverse transcription of total cellular RNA. RNA can be fragmented by methods known in the art, e.g., by incubation with ZnCl2, to generate fragments of RNA.

In one aspect, total RNA, mRNAs, or nucleic acids derived therefrom (such as cDNA), are isolated from a sample taken from a patient having a predetermined disease. Biomarkers that are poorly expressed, in particular cells, may be enriched using amplification techniques known in the art.

As described above, the biomarker polynucleotides can be detectably labeled at one or more nucleotides. Any method known in the art may be used to label the target polynucleotides. Preferably, this labeling incorporates the label uniformly along the length of the RNA, and more preferably, the labeling is carried out at a high degree of efficiency. For example, polynucleotides can be labeled by oligo-dT primed reverse transcription. Random primers (e.g., 9-mers) can be used in reverse transcription to uniformly incorporate labeled nucleotides over the full length of the polynucleotides. Alternatively, random primers may be used in conjunction with PCR methods or T7 promoter-based in vitro transcription methods in order to amplify polynucleotides.

The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. Alternatively, the detectable label can be a radiolabeled nucleotide.

Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS). Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

As discussed above, many RNA sequencing-based methods are available. Non-limiting examples comprise, e.g. whole transcriptome RNA seq, targeted RNA seq, single cell RNA seq, total RNA sequencing, mRNA sequencing, whole transcript RNA sequencing, 3′ RNA sequencing, long RNA sequencing, direct RNA sequencing. Each of these sequencing technologies have their own way of preparing samples prior to the actual sequencing step. Depending on the sequencing technology used, amplification steps may be omitted.

As used herein, a biological sample may include a body fluid or body cell or tissue and is selected from the group comprising whole blood, serum, plasma, semen, saliva, tears, urine, fecal material, sweat, buccal smears, skin, tumor tissue, cancer cells, or a combination of one or more of thereof. Preferably, the biological sample is selected from the group comprising whole blood sample, tumor tissue sample and cancer cell sample.

The inventors conducted a study aimed at the identification of predictive and early markers of response to ICBT in patients with cancer, in particular metastatic melanoma. By performing a comprehensive, unbiased whole blood transcriptome analysis, they surprisingly revealed that
one or more genes listed in Table 1, selected among the Ribosomal biogenesis genes cluster, the TCR signaling genes cluster, the Cilia genes cluster, and the Interferon pathway genes cluster, or a combination of one or more thereof are up or down regulated thus predicting if a patient will respond to a treatment based on ICBT, whereas
one or more genes listed in Table 2, selected among the Cell Cycle genes cluster, the Jak/Stat Signaling pathway genes cluster, and the T cell/Immune tolerance regulation genes cluster, or a combination of one or more thereof, are up or down regulated thus determining if a patient is responsive to a treatment based on ICBT.

TABLE 1
595 genes for response prediction to ICBT. The column “Gene List” identifies the top
25-(best25) and the top 119- (short) gene panel. Log2FC: log2 fold change; padj: adjusted p-value.

| | | logFC | pvalue | padj | Gene | Biological | |
|---|---|---|---|---|---|---|---|
| Gene ID | Gene Symbol | DEseq | Deseq | Deseq | List | Cluster | Selected by |
| ENSG00000110057 | UNC93B1 | −0.22 | 5.32E−06 | 2.00E−02 | best25 | Interferon | Multivariant |
| ENSG00000141101 | NOB1 | 0.19 | 1.18E−04 | 1.61E−01 | best25 | Ribosomal | Univariant |
| | | | | | | Biogenesis | |
| ENSG00000143748 | NVL | 0.15 | 2.11E−04 | 1.74E−01 | best25 | Ribosomal | Univariant |
| | | | | | | Biogenesis | |
| ENSG00000130811 | EIF3G | 0.18 | 2.74E−04 | 1.78E−01 | best25 | Ribosomal | Univariant |
| | | | | | | Biogenesis | |
| ENSG00000113360 | DROSHA | 0.09 | 2.71E−03 | 3.79E−01 | best25 | Ribosomal | Univariant |
| | | | | | | Biogenesis | |
| ENSG00000169100 | SLC25A6 | 0.12 | 4.30E−03 | 3.95E−01 | best25 | Ribosomal | Multivariant |
| | | | | | | Biogenesis | |
| ENSG00000175390 | EIF3F | 0.12 | 9.03E−03 | 4.21E−01 | best25 | Ribosomal | Multivariant |
| | | | | | | Biogenesis | |
| ENSG00000100450 | GZMH | 0.09 | 1.09E−03 | 2.78E−01 | best25 | TCRsignaling | Univariant |
| ENSG00000225783 | MIAT | 0.11 | 1.17E−03 | 2.90E−01 | best25 | TCRsignaling | Univariant |
| ENSG00000008517 | IL32 | 0.11 | 2.04E−03 | 3.75E−01 | best25 | TCRsignaling | Univariant |
| ENSG00000259673 | IQCH-AS1 | 2.38 | 6.60E−07 | 9.89E−03 | best25 | | Univariant |
| ENSG00000138670 | RASGEF1B | −0.24 | 4.24E−06 | 2.00E−02 | best25 | | Multivariant |
| ENSG00000145214 | DGKQ | 0.22 | 4.55E−06 | 2.00E−02 | best25 | | Univariant |
| ENSG00000126246 | IGFLR1 | −0.13 | 1.40E−04 | 1.73E−01 | best25 | | Univariant |
| ENSG00000256576 | LINC02361 | 0.13 | 1.92E−04 | 1.74E−01 | best25 | | Multivariant |
| ENSG00000260238 | PMF1-BGLAP | 0.11 | 2.85E−04 | 1.78E−01 | best25 | | Univariant |
| ENSG00000276953 | TRBV12-4 | 0.09 | 4.59E−04 | 2.26E−01 | best25 | | Univariant |
| ENSG00000159884 | CCDC107 | 0.15 | 7.14E−04 | 2.35E−01 | best25 | | Univariant |
| ENSG00000100298 | APOBEC3H | 0.10 | 7.85E−04 | 2.45E−01 | best25 | | Multivariant |
| ENSG00000196209 | SIRPB2 | −0.15 | 8.57E−04 | 2.52E−01 | best25 | | Univariant |
| ENSG00000196247 | ZNF107 | −0.10 | 1.26E−03 | 2.90E−01 | best25 | | Multivariant |
| ENSG00000257727 | CNPY2 | 0.16 | 1.30E−03 | 2.95E−01 | best25 | | Univariant |
| ENSG00000119862 | LGALSL | −0.11 | 2.13E−03 | 3.75E−01 | best25 | | Univariant |
| ENSG00000065802 | ASB1 | 0.11 | 2.28E−03 | 3.75E−01 | best25 | | Univariant |
| ENSG00000116649 | SRM | 0.10 | 3.31E−03 | 3.85E−01 | best25 | | Univariant |
| ENSG00000179750 | APOBEC3B | −0.08 | 3.00E−04 | 1.80E−01 | short | Interferon | Univariant |
| ENSG00000168404 | MLKL | −0.13 | 5.66E−04 | 2.26E−01 | short | Interferon | Univariant |
| ENSG00000135655 | USP15 | −0.13 | 2.05E−03 | 3.75E−01 | short | Interferon | Univariant |
| ENSG00000119922 | IFIT2 | −0.08 | 2.69E−03 | 3.79E−01 | short | Interferon | Univariant |
| ENSG00000185507 | IRF7 | −0.08 | 3.46E−03 | 3.85E−01 | short | Interferon | Univariant |
| ENSG00000168062 | BATF2 | 0.00 | 1.00E+00 | 1.00E+00 | short | Interferon | Univariant |
| ENSG00000087995 | METTL2A | 0.17 | 5.38E−04 | 2.26E−01 | short | Ribosomal | Univariant |
| | | | | | | Biogenesis | |
| ENSG00000105373 | NOP53 | 0.12 | 2.17E−03 | 3.75E−01 | short | Ribosomal | Univariant |
| | | | | | | Biogenesis | |
| ENSG00000071462 | BUD23 | 0.13 | 3.65E−03 | 3.86E−01 | short | Ribosomal | Univariant |
| | | | | | | Biogenesis | |
| ENSG00000063177 | RPL18 | 0.12 | 4.18E−03 | 3.92E−01 | short | Ribosomal | Univariant |
| | | | | | | Biogenesis | |
| ENSG00000175792 | RUVBL1 | 0.12 | 8.12E−03 | 4.11E−01 | short | Ribosomal | Univariant |
| | | | | | | Biogenesis | |
| ENSG00000158526 | TSR2 | 0.13 | 8.74E−03 | 4.18E−01 | short | Ribosomal | Univariant |
| | | | | | | Biogenesis | |
| ENSG00000152234 | ATP5F1A | 0.12 | 1.11E−02 | 4.34E−01 | short | Ribosomal | Univariant |
| | | | | | | Biogenesis | |
| ENSG00000179041 | RRS1 | 0.10 | 1.38E−02 | 4.54E−01 | short | Ribosomal | Univariant |
| | | | | | | Biogenesis | |
| ENSG00000156261 | CCT8 | 0.12 | 1.50E−02 | 4.56E−01 | short | Ribosomal | Univariant |
| | | | | | | Biogenesis | |
| ENSG00000152969 | JAKMIP1 | 0.16 | 3.00E−05 | 7.50E−02 | short | TCRsignaling | Univariant |
| ENSG00000203896 | LIME1 | 0.08 | 2.59E−04 | 1.78E−01 | short | TCRsignaling | Univariant |
| ENSG00000211799 | TRAV19 | 0.07 | 1.05E−03 | 2.70E−01 | short | TCRsignaling | Univariant |
| ENSG00000076641 | PAG1 | −0.13 | 1.25E−03 | 2.90E−01 | short | TCRsignaling | Univariant |
| ENSG00000172116 | CD8B | 0.07 | 2.20E−03 | 3.75E−01 | short | TCRsignaling | Univariant |
| ENSG00000153563 | CD8A | 0.07 | 2.28E−03 | 3.75E−01 | short | TCRsignaling | Univariant |
| ENSG00000064886 | CHI3L2 | 0.08 | 3.62E−03 | 3.85E−01 | short | TCRsignaling | Univariant |
| ENSG00000087266 | SH3BP2 | −0.10 | 6.84E−03 | 4.02E−01 | short | TCRsignaling | Univariant |
| ENSG00000132613 | MTSS1L | −0.43 | 7.33E−06 | 2.20E−02 | short | | Univariant |
| ENSG00000197568 | HHLA3 | 0.19 | 6.13E−05 | 1.15E−01 | short | | Univariant |
| ENSG00000099797 | TECR | 0.19 | 8.67E−05 | 1.44E−01 | short | | Univariant |
| ENSG00000088876 | ZNF343 | 0.16 | 1.11E−04 | 1.61E−01 | short | | Univariant |
| ENSG00000144426 | NBEAL1 | 0.10 | 1.65E−04 | 1.74E−01 | short | | Univariant |
| ENSG00000116127 | ALMS1 | 0.10 | 1.83E−04 | 1.74E−01 | short | | Univariant |
| ENSG00000105447 | GRWD1 | 0.11 | 2.09E−04 | 1.74E−01 | short | | Univariant |
| ENSG00000105948 | TTC26 | −0.11 | 2.21E−04 | 1.74E−01 | short | | Univariant |
| ENSG00000152952 | PLOD2 | −0.09 | 2.33E−04 | 1.74E−01 | short | | Univariant |
| ENSG00000263843 | AC022211.2 | 0.10 | 3.68E−04 | 2.04E−01 | short | | Univariant |
| ENSG00000249476 | AC008467.1 | −0.14 | 4.85E−04 | 2.26E−01 | short | | Univariant |
| ENSG00000260643 | AC092718.3 | 0.11 | 5.39E−04 | 2.26E−01 | short | | Univariant |
| ENSG00000241404 | EGFL8 | 0.08 | 5.59E−04 | 2.26E−01 | short | | Univariant |
| ENSG00000055211 | GINM1 | −0.17 | 5.77E−04 | 2.26E−01 | short | | Univariant |
| ENSG00000211746 | TRBV19 | 0.07 | 5.88E−04 | 2.26E−01 | short | | Univariant |
| ENSG00000267365 | KCNJ2-AS1 | −0.09 | 6.09E−04 | 2.26E−01 | short | | Univariant |
| ENSG00000111728 | ST8SIA1 | 0.06 | 6.15E−04 | 2.26E−01 | short | | Univariant |
| ENSG00000272221 | AL645933.2 | −0.13 | 6.33E−04 | 2.26E−01 | short | | Univariant |
| ENSG00000120280 | CXorf21 | −0.15 | 7.20E−04 | 2.35E−01 | short | | Univariant |
| ENSG00000086065 | CHMP5 | −0.11 | 7.22E−04 | 2.35E−01 | short | | Univariant |
| ENSG00000120051 | CFAP58 | −0.11 | 7.83E−04 | 2.45E−01 | short | | Univariant |
| ENSG00000105472 | CLEC11A | −0.09 | 8.59E−04 | 2.52E−01 | short | | Univariant |
| ENSG00000105088 | OLFM2 | 0.10 | 9.16E−04 | 2.54E−01 | short | | Univariant |
| ENSG00000197561 | ELANE | −0.05 | 1.21E−03 | 2.90E−01 | short | | Univariant |
| ENSG00000197381 | ADARB1 | 0.12 | 1.47E−03 | 3.11E−01 | short | | Univariant |
| ENSG00000143740 | SNAP47 | 0.15 | 1.55E−03 | 3.18E−01 | short | | Univariant |
| ENSG00000169683 | LRRC45 | 0.12 | 1.78E−03 | 3.52E−01 | short | | Multivariant |
| ENSG00000188933 | USP32P1 | −0.08 | 2.18E−03 | 3.75E−01 | short | | Univariant |
| ENSG00000257433 | AC004241.1 | −0.11 | 2.28E−03 | 3.75E−01 | short | | Univariant |
| ENSG00000225489 | AL354707.1 | 0.11 | 2.45E−03 | 3.75E−01 | short | | Univariant |
| ENSG00000225889 | AC012368.1 | −0.12 | 2.70E−03 | 3.79E−01 | short | | Univariant |
| ENSG00000106608 | URGCP | 0.11 | 2.95E−03 | 3.85E−01 | short | | Univariant |
| ENSG00000285793 | AC125232.2 | −0.14 | 2.96E−03 | 3.85E−01 | short | | Univariant |
| ENSG00000119673 | ACOT2 | 0.11 | 3.16E−03 | 3.85E−01 | short | | Univariant |
| ENSG00000270972 | AC136475.9 | −0.07 | 3.17E−03 | 3.85E−01 | short | | Univariant |
| ENSG00000196083 | IL1RAP | −0.10 | 3.25E−03 | 3.85E−01 | short | | Univariant |
| ENSG00000189362 | NEMP2 | 0.07 | 3.41E−03 | 3.85E−01 | short | | Univariant |
| ENSG00000148154 | UGCG | −0.10 | 3.50E−03 | 3.85E−01 | short | | Univariant |
| ENSG00000156253 | RWDD2B | 0.13 | 3.59E−03 | 3.85E−01 | short | | Univariant |
| ENSG00000139405 | RITA1 | 0.12 | 3.87E−03 | 3.91E−01 | short | | Univariant |
| ENSG00000132763 | MMACHC | 0.12 | 3.88E−03 | 3.91E−01 | short | | Univariant |
| ENSG00000284606 | AC105233.5 | 0.10 | 3.97E−03 | 3.91E−01 | short | | Univariant |
| ENSG00000170468 | RIOX1 | 0.14 | 4.09E−03 | 3.91E−01 | short | | Univariant |
| ENSG00000125877 | ITPA | 0.13 | 4.14E−03 | 3.91E−01 | short | | Univariant |
| ENSG00000241288 | AC092902.2 | 0.08 | 4.57E−03 | 3.95E−01 | short | | Univariant |
| ENSG00000168778 | TCTN2 | 0.09 | 5.43E−03 | 3.95E−01 | short | | Univariant |
| ENSG00000073605 | GSDMB | 0.10 | 5.49E−03 | 3.95E−01 | short | | Univariant |
| ENSG00000144152 | FBLN7 | 0.08 | 5.88E−03 | 3.95E−01 | short | | Univariant |
| ENSG00000176597 | B3GNT5 | −0.10 | 6.12E−03 | 3.95E−01 | short | | Multivariant |
| ENSG00000167699 | GLOD4 | 0.12 | 8.41E−03 | 4.13E−01 | short | | Univariant |
| ENSG00000148331 | ASB6 | 0.12 | 9.09E−03 | 4.21E−01 | short | | Multivariant |
| ENSG00000107779 | BMPR1A | 0.08 | 1.17E−02 | 4.34E−01 | short | | Multivariant |
| ENSG00000258461 | AC012651.1 | −0.10 | 1.27E−02 | 4.52E−01 | short | | Univariant |
| ENSG00000138468 | SENP7 | −0.10 | 1.72E−02 | 4.62E−01 | short | | Univariant |
| ENSG00000166477 | LEO1 | 0.11 | 1.95E−02 | 4.64E−01 | short | | Univariant |
| ENSG00000225630 | MTND2P28 | 0.04 | 4.07E−02 | 4.85E−01 | short | | Univariant |
| ENSG00000166582 | CENPV | 0.08 | 4.50E−02 | 4.92E−01 | short | | Univariant |
| ENSG00000105426 | PTPRS | −0.06 | 4.63E−02 | 4.94E−01 | short | | Univariant |
| ENSG00000120451 | SNX19 | −0.05 | 1.29E−01 | 5.96E−01 | short | | Multivariant |
| ENSG00000168477 | TNXB | −0.03 | 2.67E−01 | 7.13E−01 | short | | Univariant |
| ENSG00000079385 | CEACAM1 | 0.00 | 1.00E+00 | 1.00E+00 | short | | Univariant |
| ENSG00000106025 | TSPAN12 | 0.00 | 1.00E+00 | 1.00E+00 | short | | Univariant |
| ENSG00000121440 | PDZRN3 | 0.00 | 1.00E+00 | 1.00E+00 | short | | Univariant |
| ENSG00000112186 | CAP2 | 0.00 | 1.00E+00 | 1.00E+00 | short | | Univariant |
| ENSG00000211806 | TRAV25 | 0.00 | 1.00E+00 | 1.00E+00 | short | | Univariant |
| ENSG00000211938 | IGHV3-7 | 0.00 | 1.00E+00 | 1.00E+00 | short | | Univariant |
| ENSG00000132481 | TRIM47 | 0.00 | 1.00E+00 | 1.00E+00 | short | | Univariant |
| ENSG00000160284 | SPATC1L | 0.00 | 1.00E+00 | 1.00E+00 | short | | Univariant |
| ENSG00000188626 | GOLGA8M | 0.00 | 1.00E+00 | 1.00E+00 | short | | Univariant |
| ENSG00000232450 | RPS2P14 | 0.00 | 1.00E+00 | 1.00E+00 | short | | Univariant |
| ENSG00000272990 | AC084036.1 | 0.00 | 1.00E+00 | 1.00E+00 | short | | Univariant |
| ENSG00000203666 | EFCAB2 | −0.06 | 3.14E−03 | 3.85E−01 | | Cilia | Univariant |
| ENSG00000151023 | ENKUR | −0.05 | 2.84E−02 | 4.70E−01 | | Cilia | Univariant |
| ENSG00000132321 | IQCA1 | 0.00 | 1.00E+00 | 1.00E+00 | | Cilia | Univariant |
| ENSG00000166578 | IQCD | 0.00 | 1.00E+00 | 1.00E+00 | | Cilia | Multivariant |
| ENSG00000138496 | PARP9 | −0.09 | 3.59E−03 | 3.85E−01 | | Interferon | Univariant |
| ENSG00000205413 | SAMD9 | −0.09 | 4.11E−03 | 3.91E−01 | | Interferon | Univariant |
| ENSG00000188313 | PLSCR1 | −0.07 | 6.80E−03 | 4.01E−01 | | Interferon | Univariant |
| ENSG00000163840 | DTX3L | −0.09 | 7.95E−03 | 4.11E−01 | | Interferon | Univariant |
| ENSG00000105939 | ZC3HAV1 | −0.09 | 8.46E−03 | 4.13E−01 | | Interferon | Univariant |
| ENSG00000152778 | IFIT5 | −0.07 | 9.11E−03 | 4.21E−01 | | Interferon | Univariant |
| ENSG00000196116 | TDRD7 | −0.11 | 9.33E−03 | 4.23E−01 | | Interferon | Univariant |
| ENSG00000078081 | LAMP3 | 0.00 | 1.00E+00 | 1.00E+00 | | Interferon | Univariant |
| ENSG00000261236 | BOP1 | 0.15 | 1.55E−03 | 3.18E−01 | | Ribosomal | Univariant |
| | | | | | | Biogenesis | |
| ENSG00000173113 | TRMT112 | 0.13 | 2.44E−03 | 3.75E−01 | | Ribosomal | Univariant |
| | | | | | | Biogenesis | |
| ENSG00000188976 | NOC2L | 0.14 | 2.79E−03 | 3.82E−01 | | Ribosomal | Univariant |
| | | | | | | Biogenesis | |
| ENSG00000105202 | FBL | 0.11 | 4.13E−03 | 3.91E−01 | | Ribosomal | Univariant |
| | | | | | | Biogenesis | |
| ENSG00000148843 | PDCD11 | 0.10 | 6.55E−03 | 3.95E−01 | | Ribosomal | Univariant |
| | | | | | | Biogenesis | |
| ENSG00000133316 | WDR74 | 0.12 | 9.49E−03 | 4.23E−01 | | Ribosomal | Univariant |
| | | | | | | Biogenesis | |
| ENSG00000171453 | POLR1C | 0.09 | 2.23E−02 | 4.66E−01 | | Ribosomal | Univariant |
| | | | | | | Biogenesis | |
| ENSG00000100129 | EIF3L | 0.08 | 4.40E−02 | 4.90E−01 | | Ribosomal | Univariant |
| | | | | | | Biogenesis | |
| ENSG00000145220 | LYAR | 0.08 | 4.54E−02 | 4.92E−01 | | Ribosomal | Univariant |
| | | | | | | Biogenesis | |
| ENSG00000145425 | RPS3A | 0.00 | 1.00E+00 | 1.00E+00 | | Ribosomal | Multivariant |
| | | | | | | Biogenesis | |
| ENSG00000095015 | MAP3K1 | −0.15 | 1.89E−03 | 3.68E−01 | | TCRsignaling | Univariant |
| ENSG00000176083 | ZNF683 | 0.06 | 4.66E−03 | 3.95E−01 | | TCRsignaling | Univariant |
| ENSG00000182866 | LCK | 0.10 | 7.02E−03 | 4.03E−01 | | TCRsignaling | Univariant |
| ENSG00000137078 | SIT1 | 0.09 | 8.51E−03 | 4.13E−01 | | TCRsignaling | Univariant |
| ENSG00000026103 | FAS | −0.08 | 8.58E−03 | 4.14E−01 | | TCRsignaling | Univariant |
| ENSG00000160185 | UBASH3A | 0.08 | 9.44E−03 | 4.23E−01 | | TCRsignaling | Univariant |
| ENSG00000277734 | TRAC | 0.08 | 1.20E−02 | 4.39E−01 | | TCRsignaling | Univariant |
| ENSG00000109943 | CRTAM | 0.08 | 1.27E−02 | 4.52E−01 | | TCRsignaling | Multivariant |
| ENSG00000211772 | TRBC2 | 0.08 | 1.40E−02 | 4.56E−01 | | TCRsignaling | Univariant |
| ENSG00000198502 | HLA-DRB5 | −0.07 | 1.66E−02 | 4.62E−01 | | TCRsignaling | Univariant |
| ENSG00000122224 | LY9 | 0.08 | 1.76E−02 | 4.62E−01 | | TCRsignaling | Univariant |
| ENSG00000198851 | CD3E | 0.08 | 1.86E−02 | 4.64E−01 | | TCRsignaling | Univariant |
| ENSG00000107485 | GATA3 | 0.08 | 1.99E−02 | 4.64E−01 | | TCRsignaling | Univariant |
| ENSG00000116824 | CD2 | 0.07 | 3.42E−02 | 4.77E−01 | | TCRsignaling | Univariant |
| ENSG00000125657 | TNFSF9 | 0.00 | 1.00E+00 | 1.00E+00 | | TCRsignaling | Univariant |
| ENSG00000155657 | TTN | 0.16 | 5.73E−05 | 1.15E−01 | | | Univariant |
| ENSG00000100154 | TTC28 | −0.10 | 1.50E−04 | 1.73E−01 | | | Univariant |
| ENSG00000186073 | C15orf41 | −0.11 | 2.64E−04 | 1.78E−01 | | | Univariant |
| ENSG00000139618 | BRCA2 | −0.07 | 3.54E−04 | 2.04E−01 | | | Univariant |
| ENSG00000176401 | EID2B | −0.11 | 4.42E−04 | 2.26E−01 | | | Univariant |
| ENSG00000113645 | WWC1 | −0.07 | 5.09E−04 | 2.26E−01 | | | Univariant |
| ENSG00000176058 | TPRN | 0.08 | 5.55E−04 | 2.26E−01 | | | Univariant |
| ENSG00000039319 | ZFYVE16 | −0.15 | 6.33E−04 | 2.26E−01 | | | Univariant |
| ENSG00000069188 | SDK2 | 0.06 | 6.68E−04 | 2.33E−01 | | | Univariant |
| ENSG00000176261 | ZBTB8OS | −0.16 | 8.29E−04 | 2.52E−01 | | | Univariant |
| ENSG00000166004 | CEP295 | −0.14 | 9.14E−04 | 2.54E−01 | | | Univariant |
| ENSG00000215483 | LINC00598 | −0.08 | 9.19E−04 | 2.54E−01 | | | Univariant |
| ENSG00000104679 | R3HCC1 | 0.16 | 9.32E−04 | 2.54E−01 | | | Univariant |
| ENSG00000184307 | ZDHHC23 | −0.09 | 9.78E−04 | 2.62E−01 | | | Univariant |
| ENSG00000266074 | BAHCC1 | −0.07 | 1.02E−03 | 2.68E−01 | | | Univariant |
| ENSG00000002822 | MAD1L1 | 0.12 | 1.22E−03 | 2.90E−01 | | | Univariant |
| ENSG00000103253 | HAGHL | 0.11 | 1.24E−03 | 2.90E−01 | | | Univariant |
| ENSG00000174306 | ZHX3 | −0.10 | 1.34E−03 | 2.95E−01 | | | Univariant |
| ENSG00000173599 | PC | 0.06 | 1.34E−03 | 2.95E−01 | | | Univariant |
| ENSG00000132849 | PATJ | 0.10 | 1.47E−03 | 3.11E−01 | | | Univariant |
| ENSG00000254126 | CD8B2 | 0.07 | 1.47E−03 | 3.11E−01 | | | Univariant |
| ENSG00000145723 | GIN1 | −0.08 | 1.63E−03 | 3.28E−01 | | | Univariant |
| ENSG00000157764 | BRAF | −0.15 | 1.64E−03 | 3.28E−01 | | | Univariant |
| ENSG00000081026 | MAGI3 | 0.07 | 1.91E−03 | 3.68E−01 | | | Univariant |
| ENSG00000104763 | ASAH1 | −0.15 | 1.97E−03 | 3.74E−01 | | | Univariant |
| ENSG00000213465 | ARL2 | 0.14 | 2.07E−03 | 3.75E−01 | | | Univariant |
| ENSG00000065613 | SLK | −0.12 | 2.27E−03 | 3.75E−01 | | | Univariant |
| ENSG00000243364 | EFNA4 | 0.15 | 2.27E−03 | 3.75E−01 | | | Univariant |
| ENSG00000178078 | STAP2 | 0.11 | 2.32E−03 | 3.75E−01 | | | Univariant |
| ENSG00000266378 | AC005224.3 | 0.08 | 2.35E−03 | 3.75E−01 | | | Univariant |
| ENSG00000266677 | AC087164.1 | −0.11 | 2.36E−03 | 3.75E−01 | | | Univariant |
| ENSG00000100181 | TPTEP1 | −0.10 | 2.39E−03 | 3.75E−01 | | | Univariant |
| ENSG00000118263 | KLF7 | −0.14 | 2.41E−03 | 3.75E−01 | | | Univariant |
| ENSG00000105072 | C19orf44 | 0.07 | 2.52E−03 | 3.79E−01 | | | Univariant |
| ENSG00000196724 | ZNF418 | −0.07 | 2.58E−03 | 3.79E−01 | | | Univariant |
| ENSG00000072195 | SPEG | 0.09 | 2.67E−03 | 3.79E−01 | | | Univariant |
| ENSG00000158050 | DUSP2 | 0.07 | 2.72E−03 | 3.79E−01 | | | Univariant |
| ENSG00000171988 | JMJD1C | −0.14 | 2.72E−03 | 3.79E−01 | | | Univariant |
| ENSG00000110711 | AIP | 0.14 | 2.72E−03 | 3.79E−01 | | | Univariant |
| ENSG00000181631 | P2RY13 | −0.13 | 2.73E−03 | 3.79E−01 | | | Univariant |
| ENSG00000140284 | SLC27A2 | −0.08 | 2.80E−03 | 3.82E−01 | | | Univariant |
| ENSG00000111911 | HINT3 | −0.15 | 2.89E−03 | 3.85E−01 | | | Univariant |
| ENSG00000104205 | SGK3 | −0.14 | 2.97E−03 | 3.85E−01 | | | Univariant |
| ENSG00000156273 | BACH1 | −0.12 | 3.01E−03 | 3.85E−01 | | | Univariant |
| ENSG00000198646 | NCOA6 | 0.11 | 3.05E−03 | 3.85E−01 | | | Univariant |
| ENSG00000163935 | SFMBT1 | 0.11 | 3.18E−03 | 3.85E−01 | | | Univariant |
| ENSG00000204947 | ZNF425 | 0.07 | 3.20E−03 | 3.85E−01 | | | Univariant |
| ENSG00000159899 | NPR2 | 0.06 | 3.24E−03 | 3.85E−01 | | | Univariant |
| ENSG00000114450 | GNB4 | −0.13 | 3.27E−03 | 3.85E−01 | | | Univariant |
| ENSG00000151466 | SCLT1 | −0.14 | 3.31E−03 | 3.85E−01 | | | Univariant |
| ENSG00000165138 | ANKS6 | 0.07 | 3.37E−03 | 3.85E−01 | | | Univariant |
| ENSG00000244383 | FAM3D-AS1 | 0.05 | 3.44E−03 | 3.85E−01 | | | Univariant |
| ENSG00000132635 | PCED1A | 0.12 | 3.47E−03 | 3.85E−01 | | | Univariant |
| ENSG00000114388 | NPRL2 | 0.14 | 3.47E−03 | 3.85E−01 | | | Univariant |
| ENSG00000157741 | UBN2 | −0.14 | 3.59E−03 | 3.85E−01 | | | Univariant |
| ENSG00000164062 | APEH | 0.13 | 3.60E−03 | 3.85E−01 | | | Univariant |
| ENSG00000170004 | CHD3 | 0.13 | 3.60E−03 | 3.85E−01 | | | Univariant |
| ENSG00000118276 | B4GALT6 | −0.09 | 3.62E−03 | 3.85E−01 | | | Univariant |
| ENSG00000185947 | ZNF267 | −0.10 | 3.62E−03 | 3.85E−01 | | | Univariant |
| ENSG00000169189 | NSMCE1 | 0.14 | 3.73E−03 | 3.91E−01 | | | Univariant |
| ENSG00000110921 | MVK | 0.11 | 3.90E−03 | 3.91E−01 | | | Univariant |
| ENSG00000253352 | TUG1 | −0.14 | 3.97E−03 | 3.91E−01 | | | Univariant |
| ENSG00000147144 | CCDC120 | 0.12 | 4.07E−03 | 3.91E−01 | | | Univariant |
| ENSG00000213533 | STIMATE | −0.11 | 4.08E−03 | 3.91E−01 | | | Univariant |
| ENSG00000137672 | TRPC6 | 0.04 | 4.09E−03 | 3.91E−01 | | | Univariant |
| ENSG00000198848 | CES1 | −0.10 | 4.10E−03 | 3.91E−01 | | | Univariant |
| ENSG00000178966 | RMI1 | −0.12 | 4.10E−03 | 3.91E−01 | | | Univariant |
| ENSG00000169682 | SPNS1 | 0.13 | 4.11E−03 | 3.91E−01 | | | Univariant |
| ENSG00000117010 | ZNF684 | −0.10 | 4.12E−03 | 3.91E−01 | | | Univariant |
| ENSG00000122643 | NT5C3A | −0.11 | 4.31E−03 | 3.95E−01 | | | Univariant |
| ENSG00000279026 | AC005225.4 | 0.11 | 4.36E−03 | 3.95E−01 | | | Univariant |
| ENSG00000259895 | AC106820.2 | 0.08 | 4.53E−03 | 3.95E−01 | | | Univariant |
| ENSG00000163006 | CCDC138 | 0.08 | 4.56E−03 | 3.95E−01 | | | Univariant |
| ENSG00000100060 | MFNG | 0.13 | 4.60E−03 | 3.95E−01 | | | Univariant |
| ENSG00000278030 | TRBV7-9 | 0.06 | 4.61E−03 | 3.95E−01 | | | Univariant |
| ENSG00000159882 | ZNF230 | −0.13 | 4.64E−03 | 3.95E−01 | | | Univariant |
| ENSG00000172493 | AFF1 | −0.10 | 4.68E−03 | 3.95E−01 | | | Univariant |
| ENSG00000005448 | WDR54 | 0.11 | 4.69E−03 | 3.95E−01 | | | Univariant |
| ENSG00000146066 | HIGD2A | 0.13 | 4.72E−03 | 3.95E−01 | | | Univariant |
| ENSG00000137414 | FAM8A1 | −0.11 | 4.72E−03 | 3.95E−01 | | | Univariant |
| ENSG00000234518 | PTGES3P1 | −0.08 | 4.76E−03 | 3.95E−01 | | | Univariant |
| ENSG00000272983 | AL117339.4 | −0.11 | 4.78E−03 | 3.95E−01 | | | Univariant |
| ENSG00000235781 | LINC02569 | −0.10 | 4.79E−03 | 3.95E−01 | | | Univariant |
| ENSG00000281357 | ARRDC3-AS1 | −0.06 | 4.82E−03 | 3.95E−01 | | | Univariant |
| ENSG00000115084 | SLC35F5 | −0.12 | 4.88E−03 | 3.95E−01 | | | Univariant |
| ENSG00000141574 | SECTM1 | −0.07 | 4.94E−03 | 3.95E−01 | | | Univariant |
| ENSG00000122786 | CALD1 | −0.07 | 4.95E−03 | 3.95E−01 | | | Univariant |
| ENSG00000132274 | TRIM22 | −0.08 | 4.95E−03 | 3.95E−01 | | | Univariant |
| ENSG00000154359 | LONRF1 | −0.11 | 4.96E−03 | 3.95E−01 | | | Univariant |
| ENSG00000149516 | MS4A3 | −0.06 | 4.97E−03 | 3.95E−01 | | | Univariant |
| ENSG00000163462 | TRIM46 | 0.08 | 4.97E−03 | 3.95E−01 | | | Univariant |
| ENSG00000171174 | RBKS | 0.10 | 4.97E−03 | 3.95E−01 | | | Univariant |
| ENSG00000250198 | LINC02199 | −0.08 | 5.01E−03 | 3.95E−01 | | | Univariant |
| ENSG00000138434 | ITPRID2 | −0.11 | 5.03E−03 | 3.95E−01 | | | Univariant |
| ENSG00000164463 | CREBRF | −0.12 | 5.05E−03 | 3.95E−01 | | | Univariant |
| ENSG00000138376 | BARD1 | −0.07 | 5.06E−03 | 3.95E−01 | | | Univariant |
| ENSG00000167005 | NUDT21 | 0.11 | 5.11E−03 | 3.95E−01 | | | Univariant |
| ENSG00000141570 | CBX8 | 0.07 | 5.13E−03 | 3.95E−01 | | | Univariant |
| ENSG00000135540 | NHSL1 | −0.07 | 5.14E−03 | 3.95E−01 | | | Univariant |
| ENSG00000089327 | FXYD5 | 0.13 | 5.17E−03 | 3.95E−01 | | | Univariant |
| ENSG00000070190 | DAPP1 | −0.11 | 5.20E−03 | 3.95E−01 | | | Univariant |
| ENSG00000278600 | AC015871.3 | −0.13 | 5.21E−03 | 3.95E−01 | | | Univariant |
| ENSG00000198700 | IPO9 | 0.10 | 5.28E−03 | 3.95E−01 | | | Univariant |
| ENSG00000140199 | SLC12A6 | −0.11 | 5.28E−03 | 3.95E−01 | | | Univariant |
| ENSG00000126705 | AHDC1 | 0.06 | 5.32E−03 | 3.95E−01 | | | Univariant |
| ENSG00000238227 | TMEM250 | 0.13 | 5.36E−03 | 3.95E−01 | | | Univariant |
| ENSG00000130066 | SAT1 | −0.12 | 5.36E−03 | 3.95E−01 | | | Univariant |
| ENSG00000101400 | SNTA1 | 0.09 | 5.42E−03 | 3.95E−01 | | | Univariant |
| ENSG00000136878 | USP20 | 0.11 | 5.49E−03 | 3.95E−01 | | | Univariant |
| ENSG00000204536 | CCHCR1 | 0.10 | 5.51E−03 | 3.95E−01 | | | Univariant |
| ENSG00000105649 | RAB3A | −0.10 | 5.56E−03 | 3.95E−01 | | | Univariant |
| ENSG00000123815 | COQ8B | −0.11 | 5.57E−03 | 3.95E−01 | | | Univariant |
| ENSG00000021574 | SPAST | −0.13 | 5.59E−03 | 3.95E−01 | | | Univariant |
| ENSG00000111912 | NCOA7 | −0.11 | 5.67E−03 | 3.95E−01 | | | Univariant |
| ENSG00000136944 | LMX1B | 0.08 | 5.67E−03 | 3.95E−01 | | | Univariant |
| ENSG00000181381 | DDX60L | −0.09 | 5.67E−03 | 3.95E−01 | | | Univariant |
| ENSG00000049323 | LTBP1 | −0.08 | 5.70E−03 | 3.95E−01 | | | Univariant |
| ENSG00000243696 | AC006254.1 | −0.08 | 5.74E−03 | 3.95E−01 | | | Univariant |
| ENSG00000099949 | LZTR1 | 0.10 | 5.75E−03 | 3.95E−01 | | | Univariant |
| ENSG00000260861 | AL049634.2 | −0.09 | 5.78E−03 | 3.95E−01 | | | Univariant |
| ENSG00000142599 | RERE | −0.11 | 5.79E−03 | 3.95E−01 | | | Univariant |
| ENSG00000175518 | UBQLNL | −0.06 | 5.88E−03 | 3.95E−01 | | | Univariant |
| ENSG00000184640 | 40057 | 0.13 | 5.88E−03 | 3.95E−01 | | | Univariant |
| ENSG00000112308 | C6orf62 | −0.13 | 5.88E−03 | 3.95E−01 | | | Univariant |
| ENSG00000198604 | BAZ1A | −0.11 | 5.89E−03 | 3.95E−01 | | | Univariant |
| ENSG00000230869 | AGAP10P | −0.05 | 5.91E−03 | 3.95E−01 | | | Univariant |
| ENSG00000170949 | ZNF160 | −0.13 | 5.93E−03 | 3.95E−01 | | | Univariant |
| ENSG00000104412 | EMC2 | −0.11 | 5.99E−03 | 3.95E−01 | | | Univariant |
| ENSG00000118514 | ALDH8A1 | −0.05 | 6.02E−03 | 3.95E−01 | | | Univariant |
| ENSG00000013375 | PGM3 | −0.07 | 6.03E−03 | 3.95E−01 | | | Univariant |
| ENSG00000250608 | NUDT16-DT | −0.05 | 6.04E−03 | 3.95E−01 | | | Univariant |
| ENSG00000178952 | TUFM | 0.10 | 6.18E−03 | 3.95E−01 | | | Univariant |
| ENSG00000163069 | SGCB | 0.10 | 6.20E−03 | 3.95E−01 | | | Univariant |
| ENSG00000133243 | BTBD2 | 0.12 | 6.21E−03 | 3.95E−01 | | | Univariant |
| ENSG00000181029 | TRAPPC5 | 0.03 | 6.23E−03 | 3.95E−01 | | | Univariant |
| ENSG00000108582 | CPD | −0.10 | 6.29E−03 | 3.95E−01 | | | Univariant |
| ENSG00000285976 | AL135905.2 | −0.13 | 6.30E−03 | 3.95E−01 | | | Univariant |
| ENSG00000104964 | AES | 0.13 | 6.31E−03 | 3.95E−01 | | | Univariant |
| ENSG00000100263 | RHBDD3 | 0.12 | 6.32E−03 | 3.95E−01 | | | Univariant |
| ENSG00000087253 | LPCAT2 | −0.09 | 6.34E−03 | 3.95E−01 | | | Univariant |
| ENSG00000104979 | C19orf53 | 0.11 | 6.35E−03 | 3.95E−01 | | | Univariant |
| ENSG00000105552 | BCAT2 | 0.12 | 6.37E−03 | 3.95E−01 | | | Univariant |
| ENSG00000007038 | PRSS21 | 0.05 | 6.42E−03 | 3.95E−01 | | | Univariant |
| ENSG00000167037 | SGSM1 | 0.11 | 6.45E−03 | 3.95E−01 | | | Univariant |
| ENSG00000118420 | UBE3D | 0.09 | 6.46E−03 | 3.95E−01 | | | Univariant |
| ENSG00000205784 | ARRDC5 | 0.11 | 6.50E−03 | 3.95E−01 | | | Univariant |
| ENSG00000221866 | PLXNA4 | −0.06 | 6.50E−03 | 3.95E−01 | | | Univariant |
| ENSG00000184545 | DUSP& | 0.06 | 6.53E−03 | 3.95E−01 | | | Univariant |
| ENSG00000162542 | TMCO4 | 0.10 | 6.54E−03 | 3.95E−01 | | | Univariant |
| ENSG00000198814 | GK | −0.08 | 6.57E−03 | 3.95E−01 | | | Univariant |
| ENSG00000073331 | ALPK1 | −0.09 | 6.58E−03 | 3.95E−01 | | | Univariant |
| ENSG00000132879 | FBXO44 | 0.09 | 6.63E−03 | 3.96E−01 | | | Univariant |
| ENSG00000111885 | MAN1A1 | −0.11 | 6.73E−03 | 3.99E−01 | | | Univariant |
| ENSG00000145012 | LPP | −0.12 | 6.74E−03 | 3.99E−01 | | | Univariant |
| ENSG00000100647 | SUSD6 | −0.12 | 6.88E−03 | 4.03E−01 | | | Univariant |
| ENSG00000156990 | RPUSD3 | 0.13 | 6.92E−03 | 4.03E−01 | | | Univariant |
| ENSG00000188107 | EYS | −0.09 | 6.96E−03 | 4.03E−01 | | | Univariant |
| ENSG00000101224 | CDC25B | 0.10 | 7.00E−03 | 4.03E−01 | | | Univariant |
| ENSG00000197620 | CXorf40A | 0.13 | 7.04E−03 | 4.03E−01 | | | Univariant |
| ENSG00000172232 | AZU1 | −0.05 | 7.04E−03 | 4.03E−01 | | | Univariant |
| ENSG00000139083 | ETV6 | −0.12 | 7.12E−03 | 4.05E−01 | | | Univariant |
| ENSG00000053371 | AKR7A2 | 0.11 | 7.16E−03 | 4.05E−01 | | | Univariant |
| ENSG00000139370 | SLC15A4 | −0.13 | 7.16E−03 | 4.05E−01 | | | Univariant |
| ENSG00000240288 | GHRLOS | 0.10 | 7.21E−03 | 4.06E−01 | | | Univariant |
| ENSG00000171617 | ENC1 | 0.08 | 7.30E−03 | 4.07E−01 | | | Univariant |
| ENSG00000196074 | SYCP2 | −0.06 | 7.30E−03 | 4.07E−01 | | | Univariant |
| ENSG00000183655 | KLHL25 | 0.06 | 7.35E−03 | 4.07E−01 | | | Univariant |
| ENSG00000073756 | PTGS2 | −0.10 | 7.40E−03 | 4.07E−01 | | | Univariant |
| ENSG00000147140 | NONO | 0.13 | 7.41E−03 | 4.07E−01 | | | Univariant |
| ENSG00000113532 | ST8SIA4 | −0.12 | 7.43E−03 | 4.07E−01 | | | Univariant |
| ENSG00000107331 | ABCA2 | 0.07 | 7.44E−03 | 4.07E−01 | | | Univariant |
| ENSG00000055955 | ITIH4 | −0.07 | 7.46E−03 | 4.07E−01 | | | Univariant |
| ENSG00000108799 | EZH1 | −0.13 | 7.49E−03 | 4.07E−01 | | | Univariant |
| ENSG00000114331 | ACAP2 | −0.12 | 7.51E−03 | 4.07E−01 | | | Univariant |
| ENSG00000132507 | EIF5A | 0.12 | 7.55E−03 | 4.07E−01 | | | Univariant |
| ENSG00000172053 | QARS | 0.12 | 7.55E−03 | 4.07E−01 | | | Univariant |
| ENSG00000165105 | RASEF | 0.06 | 7.64E−03 | 4.09E−01 | | | Univariant |
| ENSG00000183726 | TMEM50A | 0.11 | 7.68E−03 | 4.09E−01 | | | Univariant |
| ENSG00000164983 | TMEM65 | −0.13 | 7.71E−03 | 4.09E−01 | | | Univariant |
| ENSG00000273247 | AC097376.2 | −0.09 | 7.71E−03 | 4.09E−01 | | | Univariant |
| ENSG00000175073 | VCPIP1 | −0.10 | 7.72E−03 | 4.09E−01 | | | Univariant |
| ENSG00000173559 | NABP1 | −0.09 | 7.76E−03 | 4.09E−01 | | | Univariant |
| ENSG00000101246 | ARFRP1 | 0.13 | 7.84E−03 | 4.11E−01 | | | Univariant |
| ENSG00000115145 | STAM2 | −0.12 | 7.86E−03 | 4.11E−01 | | | Univariant |
| ENSG00000010256 | UQCRC1 | 0.09 | 7.91E−03 | 4.11E−01 | | | Univariant |
| ENSG00000187713 | TMEM203 | 0.13 | 7.94E−03 | 4.11E−01 | | | Univariant |
| ENSG00000160183 | TMPRSS3 | 0.05 | 7.96E−03 | 4.11E−01 | | | Univariant |
| ENSG00000111676 | ATN1 | 0.09 | 7.99E−03 | 4.11E−01 | | | Univariant |
| ENSG00000254635 | WAC-AS1 | −0.13 | 8.00E−03 | 4.11E−01 | | | Univariant |
| ENSG00000104897 | SF3A2 | 0.11 | 8.04E−03 | 4.11E−01 | | | Univariant |
| ENSG00000184436 | THAP7 | 0.09 | 8.14E−03 | 4.11E−01 | | | Univariant |
| ENSG00000013275 | PSMC4 | 0.13 | 8.15E−03 | 4.11E−01 | | | Univariant |
| ENSG00000205089 | CCNI2 | 0.05 | 8.18E−03 | 4.11E−01 | | | Univariant |
| ENSG00000198898 | CAPZA2 | −0.09 | 8.18E−03 | 4.11E−01 | | | Univariant |
| ENSG00000116977 | LGALS8 | −0.11 | 8.21E−03 | 4.11E−01 | | | Univariant |
| ENSG00000230733 | AC092171.2 | 0.09 | 8.23E−03 | 4.11E−01 | | | Univariant |
| ENSG00000170545 | SMAGP | 0.11 | 8.26E−03 | 4.11E−01 | | | Univariant |
| ENSG00000265118 | AC134669.1 | −0.10 | 8.34E−03 | 4.13E−01 | | | Univariant |
| ENSG00000164530 | PI16 | 0.07 | 8.38E−03 | 4.13E−01 | | | Univariant |
| ENSG00000253616 | AC107959.3 | −0.10 | 8.40E−03 | 4.13E−01 | | | Univariant |
| ENSG00000111850 | SMIM8 | −0.10 | 8.48E−03 | 4.13E−01 | | | Univariant |
| ENSG00000211810 | TRAV29DV5 | 0.06 | 8.51E−03 | 4.13E−01 | | | Univariant |
| ENSG00000072506 | HSD17B10 | 0.12 | 8.58E−03 | 4.14E−01 | | | Univariant |
| ENSG00000070759 | TESK2 | −0.11 | 8.69E−03 | 4.17E−01 | | | Univariant |
| ENSG00000261832 | AC138894.1 | 0.07 | 8.75E−03 | 4.18E−01 | | | Univariant |
| ENSG00000165282 | PIGO | 0.10 | 8.85E−03 | 4.21E−01 | | | Univariant |
| ENSG00000211955 | IGHV3-33 | −0.06 | 8.92E−03 | 4.21E−01 | | | Univariant |
| ENSG00000285258 | ATXN7 | −0.10 | 8.97E−03 | 4.21E−01 | | | Univariant |
| ENSG00000077312 | SNRPA | 0.12 | 8.97E−03 | 4.21E−01 | | | Univariant |
| ENSG00000171861 | MRM3 | 0.11 | 9.02E−03 | 4.21E−01 | | | Univariant |
| ENSG00000117877 | CD3EAP | 0.12 | 9.06E−03 | 4.21E−01 | | | Univariant |
| ENSG00000152270 | PDE3B | −0.12 | 9.10E−03 | 4.21E−01 | | | Univariant |
| ENSG00000108961 | RANGRF | 0.11 | 9.26E−03 | 4.23E−01 | | | Univariant |
| ENSG00000166225 | FRS2 | −0.11 | 9.26E−03 | 4.23E−01 | | | Univariant |
| ENSG00000211694 | TRGV10 | 0.06 | 9.34E−03 | 4.23E−01 | | | Univariant |
| ENSG00000105750 | ZNF85 | −0.07 | 9.34E−03 | 4.23E−01 | | | Univariant |
| ENSG00000221963 | APOL6 | −0.07 | 9.34E−03 | 4.23E−01 | | | Univariant |
| ENSG00000251660 | NA | −0.07 | 9.40E−03 | 4.23E−01 | | | Univariant |
| ENSG00000182095 | TNRC18 | −0.10 | 9.51E−03 | 4.23E−01 | | | Univariant |
| ENSG00000135631 | RAB11FIP5 | 0.09 | 9.52E−03 | 4.23E−01 | | | Univariant |
| ENSG00000121749 | TBC1D15 | −0.12 | 9.54E−03 | 4.23E−01 | | | Univariant |
| ENSG00000173653 | RCE1 | 0.12 | 9.54E−03 | 4.23E−01 | | | Univariant |
| ENSG00000062716 | VMP1 | −0.11 | 9.55E−03 | 4.23E−01 | | | Univariant |
| ENSG00000024048 | UBR2 | −0.12 | 9.56E−03 | 4.23E−01 | | | Univariant |
| ENSG00000248544 | AC008676.1 | 0.08 | 9.60E−03 | 4.23E−01 | | | Univariant |
| ENSG00000185946 | RNPC3 | −0.12 | 9.68E−03 | 4.24E−01 | | | Univariant |
| ENSG00000127616 | SMARCA4 | 0.10 | 9.69E−03 | 4.24E−01 | | | Univariant |
| ENSG00000105568 | PPP2R1A | 0.09 | 9.69E−03 | 4.24E−01 | | | Univariant |
| ENSG00000105341 | DMAC2 | 0.12 | 9.81E−03 | 4.27E−01 | | | Univariant |
| ENSG00000169752 | NRG4 | −0.04 | 9.84E−03 | 4.28E−01 | | | Univariant |
| ENSG00000277511 | NA | −0.07 | 9.90E−03 | 4.29E−01 | | | Univariant |
| ENSG00000136243 | NUPL2 | 0.09 | 9.95E−03 | 4.29E−01 | | | Univariant |
| ENSG00000140829 | DHX38 | 0.10 | 9.99E−03 | 4.29E−01 | | | Univariant |
| ENSG00000178605 | GTPBP6 | 0.12 | 1.01E−02 | 4.29E−01 | | | Multivariant |
| ENSG00000231177 | LINC00852 | 0.11 | 1.02E−02 | 4.29E−01 | | | Univariant |
| ENSG00000135624 | CCT7 | 0.12 | 1.04E−02 | 4.29E−01 | | | Univariant |
| ENSG00000074071 | MRPS34 | 0.11 | 1.05E−02 | 4.29E−01 | | | Multivariant |
| ENSG00000118322 | ATP10B | 0.08 | 1.07E−02 | 4.30E−01 | | | Univariant |
| ENSG00000135069 | PSAT1 | 0.08 | 1.08E−02 | 4.31E−01 | | | Univariant |
| ENSG00000163344 | PMVK | 0.11 | 1.10E−02 | 4.34E−01 | | | Multivariant |
| ENSG00000079482 | OPHN1 | 0.10 | 1.13E−02 | 4.34E−01 | | | Univariant |
| ENSG00000125457 | MIF4GD | 0.12 | 1.15E−02 | 4.34E−01 | | | Multivariant |
| ENSG00000070756 | PABPC1 | 0.12 | 1.15E−02 | 4.34E−01 | | | Univariant |
| ENSG00000144655 | CSRNP1 | −0.10 | 1.17E−02 | 4.34E−01 | | | Univariant |
| ENSG00000100226 | GTPBP1 | −0.08 | 1.35E−02 | 4.54E−01 | | | Univariant |
| ENSG00000130511 | SSBP4 | 0.10 | 1.39E−02 | 4.54E−01 | | | Univariant |
| ENSG00000226950 | DANCR | 0.10 | 1.53E−02 | 4.56E−01 | | | Univariant |
| ENSG00000239789 | MRPS17 | 0.09 | 1.59E−02 | 4.57E−01 | | | Univariant |
| ENSG00000142230 | SAE1 | 0.11 | 1.65E−02 | 4.62E−01 | | | Univariant |
| ENSG00000146426 | TIAM2 | −0.09 | 1.80E−02 | 4.62E−01 | | | Univariant |
| ENSG00000248019 | FAM13A-AS1 | −0.11 | 1.83E−02 | 4.63E−01 | | | Multivariant |
| ENSG00000255328 | AC136475.5 | −0.06 | 1.89E−02 | 4.64E−01 | | | Univariant |
| ENSG00000110031 | LPXN | 0.11 | 1.94E−02 | 4.64E−01 | | | Univariant |
| ENSG00000125772 | GPCPD1 | −0.11 | 1.95E−02 | 4.64E−01 | | | Multivariant |
| ENSG00000159692 | CTBP1 | 0.11 | 2.22E−02 | 4.66E−01 | | | Univariant |
| ENSG00000246089 | AC016065.1 | 0.07 | 2.26E−02 | 4.66E−01 | | | Univariant |
| ENSG00000187601 | MAGEH1 | 0.09 | 2.39E−02 | 4.67E−01 | | | Univariant |
| ENSG00000227678 | AL355581.1 | 0.07 | 2.59E−02 | 4.70E−01 | | | Univariant |
| ENSG00000251158 | AC131392.2 | −0.05 | 2.75E−02 | 4.70E−01 | | | Univariant |
| ENSG00000214941 | ZSWIM7 | 0.07 | 2.80E−02 | 4.70E−01 | | | Multivariant |
| ENSG00000181588 | MEX3D | 0.09 | 2.88E−02 | 4.70E−01 | | | Univariant |
| ENSG00000242071 | RPL7AP6 | 0.09 | 2.90E−02 | 4.70E−01 | | | Multivariant |
| ENSG00000104936 | DMPK | 0.07 | 3.00E−02 | 4.70E−01 | | | Univariant |
| ENSG00000162739 | SLAMF6 | 0.08 | 3.08E−02 | 4.71E−01 | | | Univariant |
| ENSG00000082996 | RNF13 | −0.09 | 3.12E−02 | 4.72E−01 | | | Multivariant |
| ENSG00000135486 | HNRNPA1 | 0.10 | 3.16E−02 | 4.72E−01 | | | Univariant |
| ENSG00000160563 | MED27 | 0.10 | 3.26E−02 | 4.74E−01 | | | Multivariant |
| ENSG00000272410 | NA | −0.03 | 3.28E−02 | 4.74E−01 | | | Univariant |
| ENSG00000232931 | LINC00342 | 0.08 | 3.35E−02 | 4.77E−01 | | | Univariant |
| ENSG00000136104 | RNASEH2B | 0.09 | 3.45E−02 | 4.78E−01 | | | Univariant |
| ENSG00000166136 | NDUFB8 | 0.10 | 3.47E−02 | 4.78E−01 | | | Multivariant |
| ENSG00000171953 | ATPAF2 | 0.09 | 3.51E−02 | 4.79E−01 | | | Multivariant |
| ENSG00000178425 | NT5DC1 | 0.08 | 3.54E−02 | 4.79E−01 | | | Univariant |
| ENSG00000285535 | AC021683.5 | 0.04 | 3.64E−02 | 4.80E−01 | | | Univariant |
| ENSG00000235560 | AC002310.1 | 0.08 | 3.67E−02 | 4.80E−01 | | | Multivariant |
| ENSG00000187837 | HIST1H1C | −0.06 | 3.96E−02 | 4.83E−01 | | | Multivariant |
| ENSG00000267520 | AC010733.2 | −0.09 | 4.48E−02 | 4.92E−01 | | | Multivariant |
| ENSG00000010818 | HIVEP2 | −0.07 | 4.52E−02 | 4.92E−01 | | | Multivariant |
| ENSG00000277701 | AC159540.2 | 0.06 | 4.68E−02 | 4.95E−01 | | | Univariant |
| ENSG00000143436 | MRPL9 | 0.08 | 4.86E−02 | 4.96E−01 | | | Univariant |
| ENSG00000160752 | FDPS | 0.09 | 4.88E−02 | 4.96E−01 | | | Multivariant |
| ENSG00000132970 | WASF3 | −0.05 | 5.01E−02 | 5.02E−01 | | | Univariant |
| ENSG00000254413 | CHKB-CPT1B | −0.05 | 5.38E−02 | 5.09E−01 | | | Univariant |
| ENSG00000100216 | TOMM22 | 0.09 | 5.53E−02 | 5.12E−01 | | | Multivariant |
| ENSG00000147684 | NDUFB9 | 0.08 | 6.20E−02 | 5.21E−01 | | | Multivariant |
| ENSG00000176171 | BNIP3 | 0.07 | 6.42E−02 | 5.26E−01 | | | Univariant |
| ENSG00000144867 | SRPRB | 0.08 | 6.47E−02 | 5.27E−01 | | | Univariant |
| ENSG00000084092 | NOA1 | 0.09 | 6.65E−02 | 5.30E−01 | | | Multivariant |
| ENSG00000084676 | NCOA1 | −0.08 | 6.77E−02 | 5.30E−01 | | | Multivariant |
| ENSG00000131503 | ANKHD1 | −0.08 | 6.87E−02 | 5.32E−01 | | | Multivariant |
| ENSG00000214193 | SH3D21 | −0.05 | 7.05E−02 | 5.36E−01 | | | Univariant |
| ENSG00000129933 | MAU2 | −0.08 | 7.35E−02 | 5.41E−01 | | | Multivariant |
| ENSG00000284428 | IPO5P1 | 0.07 | 7.36E−02 | 5.41E−01 | | | Univariant |
| ENSG00000175575 | PAAF1 | 0.07 | 7.75E−02 | 5.47E−01 | | | Univariant |
| ENSG00000133317 | LGALS12 | 0.05 | 7.77E−02 | 5.47E−01 | | | Multivariant |
| ENSG00000132773 | TOE1 | 0.07 | 9.24E−02 | 5.66E−01 | | | Multivariant |
| ENSG00000165055 | METTL2B | 0.07 | 9.40E−02 | 5.66E−01 | | | Univariant |
| ENSG00000109445 | ZNF330 | 0.08 | 9.57E−02 | 5.67E−01 | | | Univariant |
| ENSG00000181924 | COA4 | 0.07 | 9.64E−02 | 5.67E−01 | | | Multivariant |
| ENSG00000089505 | CMTM1 | −0.05 | 1.04E−01 | 5.71E−01 | | | Multivariant |
| ENSG00000129667 | RHBDF2 | −0.06 | 1.08E−01 | 5.75E−01 | | | Univariant |
| ENSG00000170248 | PDCD6IP | −0.06 | 1.15E−01 | 5.84E−01 | | | Multivariant |
| ENSG00000111639 | MRPL51 | 0.06 | 1.24E−01 | 5.91E−01 | | | Multivariant |
| ENSG00000128524 | ATP6V1F | 0.07 | 1.24E−01 | 5.91E−01 | | | Multivariant |
| ENSG00000184047 | DIABLO | 0.07 | 1.30E−01 | 5.97E−01 | | | Multivariant |
| ENSG00000139631 | CSAD | −0.06 | 1.59E−01 | 6.23E−01 | | | Multivariant |
| ENSG00000113068 | PFDN1 | 0.06 | 1.72E−01 | 6.39E−01 | | | Multivariant |
| ENSG00000166199 | ALKBH3 | 0.05 | 2.26E−01 | 6.81E−01 | | | Multivariant |
| ENSG00000140443 | IGF1R | −0.04 | 2.29E−01 | 6.82E−01 | | | Multivariant |
| ENSG00000071967 | CYBRD1 | −0.04 | 2.48E−01 | 6.99E−01 | | | Multivariant |
| ENSG00000033627 | ATP6V0A1 | −0.05 | 2.59E−01 | 7.06E−01 | | | Multivariant |
| ENSG00000236445 | LINC00608 | −0.01 | 2.68E−01 | 7.14E−01 | | | Univariant |
| ENSG00000090924 | PLEKHG2 | −0.03 | 2.86E−01 | 7.26E−01 | | | Multivariant |
| ENSG00000139990 | DCAF5 | −0.04 | 4.26E−01 | 8.01E−01 | | | Multivariant |
| ENSG00000180228 | PRKRA | 0.04 | 4.27E−01 | 8.02E−01 | | | Multivariant |
| ENSG00000230629 | RPS23P8 | 0.02 | 5.04E−01 | 8.41E−01 | | | Multivariant |
| ENSG00000104695 | PPP2CB | 0.03 | 5.48E−01 | 8.62E−01 | | | Multivariant |
| ENSG00000204237 | OXLD1 | −0.02 | 5.61E−01 | 8.70E−01 | | | Multivariant |
| ENSG00000132768 | DPH2 | 0.02 | 6.09E−01 | 8.88E−01 | | | Multivariant |
| ENSG00000107736 | CDH23 | −0.02 | 6.28E−01 | 8.94E−01 | | | Univariant |
| ENSG00000133773 | CCDC59 | 0.02 | 6.29E−01 | 8.95E−01 | | | Multivariant |
| ENSG00000064270 | ATP2C2 | −0.01 | 6.89E−01 | 9.17E−01 | | | Univariant |
| ENSG00000070476 | ZXDC | −0.02 | 6.91E−01 | 9.17E−01 | | | Multivariant |
| ENSG00000179144 | GIMAP7 | 0.01 | 8.04E−01 | 9.55E−01 | | | Multivariant |
| ENSG00000175768 | TOMM5 | −0.01 | 8.41E−01 | 9.64E−01 | | | Multivariant |
| ENSG00000198168 | SVIP | 0.00 | 8.76E−01 | 9.70E−01 | | | Multivariant |
| ENSG00000077152 | UBE2T | 0.00 | 8.84E−01 | 9.72E−01 | | | Multivariant |
| ENSG00000118680 | MYL12B | 0.00 | 9.12E−01 | 9.81E−01 | | | Multivariant |
| ENSG00000118620 | ZNF430 | 0.00 | 9.33E−01 | 9.86E−01 | | | Multivariant |
| ENSG00000178719 | GRINA | 0.00 | 9.51E−01 | 9.89E−01 | | | Multivariant |
| ENSG00000123131 | PRDX4 | 0.00 | 9.64E−01 | 9.93E−01 | | | Multivariant |
| ENSG00000268903 | AL627309.6 | 0.00 | 1.00E+00 | 1.00E+00 | | | Multivariant |
| ENSG00000106538 | RARRES2 | 0.00 | 1.00E+00 | 1.00E+00 | | | Univariant |
| ENSG00000132688 | NES | 0.00 | 1.00E+00 | 1.00E+00 | | | Univariant |
| ENSG00000250056 | LINC01018 | 0.00 | 1.00E+00 | 1.00E+00 | | | Univariant |
| ENSG00000103449 | SALL1 | 0.00 | 1.00E+00 | 1.00E+00 | | | Univariant |
| ENSG00000106511 | MEOX2 | 0.00 | 1.00E+00 | 1.00E+00 | | | Univariant |
| ENSG00000116132 | PRRX1 | 0.00 | 1.00E+00 | 1.00E+00 | | | Univariant |
| ENSG00000123560 | PLP1 | 0.00 | 1.00E+00 | 1.00E+00 | | | Univariant |
| ENSG00000143171 | RXRG | 0.00 | 1.00E+00 | 1.00E+00 | | | Univariant |
| ENSG00000188153 | COL4A5 | 0.00 | 1.00E+00 | 1.00E+00 | | | Univariant |
| ENSG00000202198 | RN7SK | 0.00 | 1.00E+00 | 1.00E+00 | | | Univariant |
| ENSG00000211448 | DIO2 | 0.00 | 1.00E+00 | 1.00E+00 | | | Univariant |
| ENSG00000211777 | TRAV3 | 0.00 | 1.00E+00 | 1.00E+00 | | | Univariant |
| ENSG00000211796 | TRAV16 | 0.00 | 1.00E+00 | 1.00E+00 | | | Univariant |
| ENSG00000241755 | GKV1-9 | 0.00 | 1.00E+00 | 1.00E+00 | | | Univariant |
| ENSG00000244437 | IGKV3-15 | 0.00 | 1.00E+00 | 1.00E+00 | | | Univariant |
| ENSG00000249751 | ECSCR | 0.00 | 1.00E+00 | 1.00E+00 | | | Univariant |
| ENSG00000088367 | EPB41L1 | 0.00 | 1.00E+00 | 1.00E+00 | | | Univariant |
| ENSG00000124479 | NDP | 0.00 | 1.00E+00 | 1.00E+00 | | | Univariant |
| ENSG00000128052 | KDR | 0.00 | 1.00E+00 | 1.00E+00 | | | Univariant |
| ENSG00000179954 | SSC5D | 0.00 | 1.00E+00 | 1.00E+00 | | | Univariant |
| ENSG00000244649 | LINC02086 | 0.00 | 1.00E+00 | 1.00E+00 | | | Univariant |
| ENSG00000120327 | PCDHB14 | 0.00 | 1.00E+00 | 1.00E+00 | | | Univariant |
| ENSG00000130224 | LRCH2 | 0.00 | 1.00E+00 | 1.00E+00 | | | Univariant |
| ENSG00000153707 | PTPRD | 0.00 | 1.00E+00 | 1.00E+00 | | | Univariant |
| ENSG00000156103 | MMP16 | 0.00 | 1.00E+00 | 1.00E+00 | | | Univariant |
| ENSG00000163873 | GRIK3 | 0.00 | 1.00E+00 | 1.00E+00 | | | Univariant |
| ENSG00000187678 | SPRY4 | 0.00 | 1.00E+00 | 1.00E+00 | | | Univariant |
| ENSG00000188338 | SLC38A3 | 0.00 | 1.00E+00 | 1.00E+00 | | | Univariant |
| ENSG00000189056 | RELN | 0.00 | 1.00E+00 | 1.00E+00 | | | Univariant |
| ENSG00000272636 | DOC2B | 0.00 | 1.00E+00 | 1.00E+00 | | | Univariant |
| ENSG00000152463 | OLAH | 0.00 | 1.00E+00 | 1.00E+00 | | | Univariant |
| ENSG00000177694 | NAALADL2 | 0.00 | 1.00E+00 | 1.00E+00 | | | Univariant |
| ENSG00000224183 | SDHDP6 | 0.00 | 1.00E+00 | 1.00E+00 | | | Univariant |
| ENSG00000225523 | IGKV6D-21 | 0.00 | 1.00E+00 | 1.00E+00 | | | Univariant |
| ENSG00000101333 | PLCB4 | 0.00 | 1.00E+00 | 1.00E+00 | | | Univariant |
| ENSG00000157554 | ERG | 0.00 | 1.00E+00 | 1.00E+00 | | | Univariant |
| ENSG00000183579 | ZNRF3 | 0.00 | 1.00E+00 | 1.00E+00 | | | Univariant |
| ENSG00000224940 | PRRT4 | 0.00 | 1.00E+00 | 1.00E+00 | | | Univariant |
| ENSG00000010030 | ETV7 | 0.00 | 1.00E+00 | 1.00E+00 | | | Univariant |
| ENSG00000110169 | HPX | 0.00 | 1.00E+00 | 1.00E+00 | | | Univariant |
| ENSG00000133063 | CHIT1 | 0.00 | 1.00E+00 | 1.00E+00 | | | Univariant |
| ENSG00000167733 | HSD11B1L | 0.00 | 1.00E+00 | 1.00E+00 | | | Univariant |
| ENSG00000171224 | FAM241B | 0.00 | 1.00E+00 | 1.00E+00 | | | Univariant |
| ENSG00000196169 | KIF19 | 0.00 | 1.00E+00 | 1.00E+00 | | | Univariant |
| ENSG00000215533 | LINC00189 | 0.00 | 1.00E+00 | 1.00E+00 | | | Univariant |
| ENSG00000225964 | NRIR | 0.00 | 1.00E+00 | 1.00E+00 | | | Univariant |
| ENSG00000267265 | AC011476.3 | 0.00 | 1.00E+00 | 1.00E+00 | | | Univariant |
| ENSG00000275158 | TRBV12-5 | 0.00 | 1.00E+00 | 1.00E+00 | | | Univariant |
| ENSG00000275601 | AC011330.2 | 0.00 | 1.00E+00 | 1.00E+00 | | | Univariant |
| ENSG00000284523 | AC004834.1 | 0.00 | 1.00E+00 | 1.00E+00 | | | Univariant |
| ENSG00000003137 | CYP26B1 | 0.00 | 1.00E+00 | 1.00E+00 | | | Univariant |
| ENSG00000102452 | NALCN | 0.00 | 1.00E+00 | 1.00E+00 | | | Univariant |
| ENSG00000107159 | CA9 | 0.00 | 1.00E+00 | 1.00E+00 | | | Univariant |
| ENSG00000112183 | RBM24 | 0.00 | 1.00E+00 | 1.00E+00 | | | Univariant |
| ENSG00000112981 | NME5 | 0.00 | 1.00E+00 | 1.00E+00 | | | Univariant |
| ENSG00000113248 | PCDHB15 | 0.00 | 1.00E+00 | 1.00E+00 | | | Univariant |
| ENSG00000116254 | CHD5 | 0.00 | 1.00E+00 | 1.00E+00 | | | Univariant |
| ENSG00000141441 | GAREM1 | 0.00 | 1.00E+00 | 1.00E+00 | | | Univariant |
| ENSG00000170745 | KCNS3 | 0.00 | 1.00E+00 | 1.00E+00 | | | Univariant |
| ENSG00000171243 | SOSTDC1 | 0.00 | 1.00E+00 | 1.00E+00 | | | Univariant |
| ENSG00000173376 | NDNF | 0.00 | 1.00E+00 | 1.00E+00 | | | Univariant |
| ENSG00000179111 | HES7 | 0.00 | 1.00E+00 | 1.00E+00 | | | Univariant |
| ENSG00000184254 | ALDH1A3 | 0.00 | 1.00E+00 | 1.00E+00 | | | Univariant |
| ENSG00000066248 | NGER | 0.00 | 1.00E+00 | 1.00E+00 | | | Univariant |
| ENSG00000091136 | LAMB1 | 0.00 | 1.00E+00 | 1.00E+00 | | | Univariant |
| ENSG00000100146 | SOX10 | 0.00 | 1.00E+00 | 1.00E+00 | | | Univariant |
| ENSG00000102287 | GABRE | 0.00 | 1.00E+00 | 1.00E+00 | | | Univariant |
| ENSG00000103710 | RASL12 | 0.00 | 1.00E+00 | 1.00E+00 | | | Univariant |
| ENSG00000123201 | GUCY1B2 | 0.00 | 1.00E+00 | 1.00E+00 | | | Univariant |
| ENSG00000124194 | GDAP1L1 | 0.00 | 1.00E+00 | 1.00E+00 | | | Univariant |
| ENSG00000132429 | POPDC3 | 0.00 | 1.00E+00 | 1.00E+00 | | | Univariant |
| ENSG00000137558 | PI15 | 0.00 | 1.00E+00 | 1.00E+00 | | | Univariant |
| ENSG00000137561 | TTPA | 0.00 | 1.00E+00 | 1.00E+00 | | | Univariant |
| ENSG00000146013 | GFRA3 | 0.00 | 1.00E+00 | 1.00E+00 | | | Univariant |
| ENSG00000162814 | SPATA17 | 0.00 | 1.00E+00 | 1.00E+00 | | | Univariant |
| ENSG00000163638 | ADAMTS9 | 0.00 | 1.00E+00 | 1.00E+00 | | | Univariant |
| ENSG00000164647 | STEAP1 | 0.00 | 1.00E+00 | 1.00E+00 | | | Univariant |
| ENSG00000164684 | ZNF704 | 0.00 | 1.00E+00 | 1.00E+00 | | | Univariant |
| ENSG00000165495 | PKNOX2 | 0.00 | 1.00E+00 | 1.00E+00 | | | Univariant |
| ENSG00000166455 | C16orf46 | 0.00 | 1.00E+00 | 1.00E+00 | | | Univariant |
| ENSG00000168621 | GDNF | 0.00 | 1.00E+00 | 1.00E+00 | | | Univariant |
| ENSG00000185551 | NR2F2 | 0.00 | 1.00E+00 | 1.00E+00 | | | Univariant |
| ENSG00000187987 | ZSCAN 23 | 0.00 | 1.00E+00 | 1.00E+00 | | | Univariant |
| ENSG00000188643 | S100A16 | 0.00 | 1.00E+00 | 1.00E+00 | | | Univariant |
| ENSG00000197360 | ZNF98 | 0.00 | 1.00E+00 | 1.00E+00 | | | Univariant |
| ENSG00000212864 | RNF208 | 0.00 | 1.00E+00 | 1.00E+00 | | | Univariant |
| ENSG00000215146 | BX322639.1 | 0.00 | 1.00E+00 | 1.00E+00 | | | Univariant |
| ENSG00000224945 | AL353150.1 | 0.00 | 1.00E+00 | 1.00E+00 | | | Univariant |
| ENSG00000249853 | HS3ST5 | 0.00 | 1.00E+00 | 1.00E+00 | | | Univariant |
| ENSG00000026036 | RTEL1- | 0.00 | 1.00E+00 | 1.00E+00 | | | Multivariant |
| | TNFRSF6B | | | | | | |
| ENSG00000164542 | KIAA0895 | 0.00 | 1.00E+00 | 1.00E+00 | | | Multivariant |
| ENSG00000205213 | LGR4 | 0.00 | 1.00E+00 | 1.00E+00 | | | Multivariant |
| ENSG00000060656 | PTPRU | 0.00 | 1.00E+00 | 1.00E+00 | | | Univariant |
| ENSG00000089356 | FXYD3 | 0.00 | 1.00E+00 | 1.00E+00 | | | Univariant |
| ENSG00000102024 | PLS3 | 0.00 | 1.00E+00 | 1.00E+00 | | | Univariant |
| ENSG00000134508 | CABLES1 | 0.00 | 1.00E+00 | 1.00E+00 | | | Univariant |
| ENSG00000138336 | TET1 | 0.00 | 1.00E+00 | 1.00E+00 | | | Univariant |
| ENSG00000153291 | SLC25A27 | 0.00 | 1.00E+00 | 1.00E+00 | | | Univariant |
| ENSG00000154493 | C10orf90 | 0.00 | 1.00E+00 | 1.00E+00 | | | Univariant |
| ENSG00000157851 | DPYSL5 | 0.00 | 1.00E+00 | 1.00E+00 | | | Univariant |
| ENSG00000175906 | ARL4D | 0.00 | 1.00E+00 | 1.00E+00 | | | Univariant |
| ENSG00000186340 | THBS2 | 0.00 | 1.00E+00 | 1.00E+00 | | | Univariant |
| ENSG00000197847 | SLC22A20P | 0.00 | 1.00E+00 | 1.00E+00 | | | Univariant |
| ENSG00000260604 | AL590004.3 | 0.00 | 1.00E+00 | 1.00E+00 | | | Univariant |
| ENSG00000268460 | AC006262.1 | 0.00 | 1.00E+00 | 1.00E+00 | | | Univariant |
| ENSG00000279329 | AC020910.5 | 0.00 | 1.00E+00 | 1.00E+00 | | | Univariant |
| ENSG00000279970 | AC023024.2 | 0.00 | 1.00E+00 | 1.00E+00 | | | Univariant |

TABLE 2
388 genes of early response. The column “Gene List” identifies the top 25- (best25)
and the top 141- (short) gene panel. log2FC: log2 fold change; padj: adjusted p-value.

| | Gene | logFC | pvalue | padj | | Biological | |
|---|---|---|---|---|---|---|---|
| Gene ID | Symbol | DEseq | Deseq | Deseq | Gene List | Cluster | Selected by |
| ENSG00000157456 | CCNB2 | 1.63 | 7.73E−10 | 2.15E−06 | best25 | CellCycle | Multivariant |
| ENSG00000176890 | TYMS | 1.48 | 1.26E−08 | 1.59E−05 | best25 | CellCycle | Multivariant |
| ENSG00000164611 | PTTG1 | 1.27 | 5.47E−10 | 2.15E−06 | best25 | CellCycle | Multivariant |
| ENSG00000148773 | MKI67 | 1.34 | 3.84E−06 | 1.33E−03 | best25 | CellCycle | Multivariant |
| ENSG00000171848 | RRM2 | 1.85 | 8.52E−10 | 2.15E−06 | best25 | CellCycle | Multivariant |
| ENSG00000109805 | NCAPG | 1.88 | 3.14E−09 | 4.75E−06 | best25 | CellCycle | Multivariant |
| ENSG00000144354 | CDCA7 | 1.47 | 9.82E−12 | 1.49E−07 | best25 | CellCycle | Multivariant |
| ENSG00000166483 | WEE1 | 0.85 | 1.13E−06 | 5.51E−04 | best25 | CellCycle | Multivariant |
| ENSG00000117399 | CDC20 | 1.80 | 2.16E−10 | 1.63E−06 | best25 | CellCycle | Multivariant |
| ENSG00000138180 | CEP55 | 1.72 | 1.13E−09 | 2.45E−06 | best25 | CellCycle | Multivariant |
| ENSG00000162063 | CCNF | 0.54 | 1.25E−05 | 3.24E−03 | best25 | CellCycle | Multivariant |
| ENSG00000088325 | TPX2 | 1.33 | 2.25E−07 | 1.62E−04 | best25 | CellCycle | Multivariant |
| ENSG00000188389 | PDCD1 | 1.47 | 5.25E−08 | 4.18E−05 | best25 | Tcell | Multivariant |
| ENSG00000163599 | CTLA4 | 1.21 | 3.67E−08 | 3.09E−05 | best25 | Tcell | Multivariant |
| ENSG00000183813 | CCR4 | 0.61 | 1.84E−05 | 4.23E−03 | best25 | Tcell | Multivariant |
| ENSG00000131203 | IDO1 | 2.27 | 2.59E−08 | 2.62E−05 | best25 | Tcell | Multivariant |
| ENSG00000131471 | AOC3 | −0.85 | 3.34E−07 | 2.02E−04 | best25 | Tcell | Multivariant |
| ENSG00000162654 | GBP4 | 1.36 | 3.86E−06 | 1.33E−03 | best25 | | Multivariant |
| ENSG00000128656 | CHN1 | 1.01 | 2.28E−06 | 8.85E−04 | best25 | | Multivariant |
| ENSG00000166803 | PCLAF | 2.15 | 6.85E−10 | 2.15E−06 | best25 | | Multivariant |
| ENSG00000163082 | SGPP2 | 1.01 | 8.26E−09 | 1.14E−05 | best25 | | Multivariant |
| ENSG00000140332 | TLE3 | −0.82 | 2.58E−07 | 1.78E−04 | best25 | | Multivariant |
| ENSG00000141753 | IGFBP4 | 0.81 | 9.76E−07 | 4.92E−04 | best25 | | Multivariant |
| ENSG00000111837 | MAK | −0.64 | 1.45E−05 | 3.53E−03 | best25 | | Multivariant |
| ENSG00000225783 | MIAT | 0.44 | 2.08E−04 | 1.92E−02 | best25 | | Multivariant |
| ENSG00000066279 | ASPM | 1.36 | 1.67E−06 | 7.14E−04 | short | CellCycle | Multivariant |
| ENSG00000134057 | CCNB1 | 1.29 | 3.37E−08 | 3.00E−05 | short | CellCycle | Multivariant |
| ENSG00000138160 | KIF11 | 1.20 | 1.58E−09 | 2.66E−06 | short | CellCycle | Univariant |
| ENSG00000145386 | CCNA2 | 1.38 | 1.61E−08 | 1.87E−05 | short | CellCycle | Univariant |
| ENSG00000122952 | ZWINT | 1.17 | 2.92E−07 | 1.84E−04 | short | CellCycle | Univariant |
| ENSG00000126787 | DLGAP5 | 1.36 | 9.13E−07 | 4.76E−04 | short | CellCycle | Univariant |
| ENSG00000106462 | EZH2 | 0.81 | 2.77E−08 | 2.62E−05 | short | CellCycle | Univariant |
| ENSG00000117632 | STMN1 | 0.61 | 2.98E−05 | 6.01E−03 | short | CellCycle | Univariant |
| ENSG00000140525 | FANCI | 0.35 | 7.18E−04 | 4.20E−02 | short | CellCycle | Multivariant |
| ENSG00000090889 | KIF4A | 0.56 | 4.06E−04 | 3.07E−02 | short | CellCycle | Multivariant |
| ENSG00000051180 | RAD51 | 1.26 | 2.47E−06 | 9.35E−04 | short | CellCycle | Univariant |
| ENSG00000146670 | CDCA5 | 1.21 | 1.44E−06 | 6.82E−04 | short | CellCycle | Univariant |
| ENSG00000170312 | CDK1 | 1.36 | 5.23E−07 | 2.82E−04 | short | CellCycle | Univariant |
| ENSG00000113810 | SMC4 | 0.41 | 4.74E−05 | 7.86E−03 | short | CellCycle | Univariant |
| ENSG00000087586 | AURKA | 0.53 | 1.49E−05 | 3.58E−03 | short | CellCycle | Univariant |
| ENSG00000035499 | DEPDC1B | 0.83 | 2.62E−05 | 5.35E−03 | short | CellCycle | Univariant |
| ENSG00000085840 | ORC1 | 0.84 | 1.15E−05 | 3.06E−03 | short | CellCycle | Univariant |
| ENSG00000112312 | GMNN | 0.73 | 1.75E−06 | 7.14E−04 | short | CellCycle | Univariant |
| ENSG00000121152 | NCAPH | 1.01 | 1.39E−05 | 3.46E−03 | short | CellCycle | Univariant |
| ENSG00000123485 | HJURP | 1.19 | 3.12E−06 | 1.12E−03 | short | CellCycle | Univariant |
| ENSG00000131747 | TOP2A | 1.10 | 7.31E−06 | 2.13E−03 | short | CellCycle | Univariant |
| ENSG00000161800 | RACGAP1 | 0.62 | 2.79E−06 | 1.03E−03 | short | CellCycle | Univariant |
| ENSG00000169679 | BUB1 | 0.97 | 9.71E−06 | 2.72E−03 | short | CellCycle | Univariant |
| ENSG00000197299 | BLM | 0.83 | 4.04E−05 | 7.14E−03 | short | CellCycle | Univariant |
| ENSG00000182628 | SKA2 | 0.34 | 6.36E−04 | 3.86E−02 | short | CellCycle | Univariant |
| ENSG00000105173 | CCNE1 | 0.49 | 1.83E−04 | 1.76E−02 | short | CellCycle | Univariant |
| ENSG00000131153 | GINS2 | 0.63 | 1.82E−04 | 1.76E−02 | short | CellCycle | Univariant |
| ENSG00000143228 | NUF2 | 0.69 | 2.19E−04 | 1.96E−02 | short | CellCycle | Univariant |
| ENSG00000072571 | HMMR | 0.00 | 1.00E+00 | 1.00E+00 | short | CellCycle | Univariant |
| ENSG00000100911 | PSME2 | 0.48 | 7.45E−05 | 1.04E−02 | short | CellCycle | Univariant |
| ENSG00000132646 | PCNA | 0.39 | 5.97E−05 | 9.12E−03 | short | CellCycle | Univariant |
| ENSG00000134308 | YWHAQ | 0.28 | 6.15E−05 | 9.22E−03 | short | CellCycle | Univariant |
| ENSG00000143106 | PSMA5 | 0.33 | 4.71E−05 | 7.86E−03 | short | CellCycle | Univariant |
| ENSG00000137804 | NUSAP1 | 0.41 | 4.88E−04 | 3.40E−02 | short | CellCycle | Univariant |
| ENSG00000185480 | PARPBP | 0.37 | 4.64E−04 | 3.31E−02 | short | CellCycle | Univariant |
| ENSG00000166888 | STAT6 | −0.30 | 8.49E−05 | 1.14E−02 | short | JAKSTAT | Multivariant |
| | | | | | | signaling | |
| ENSG00000171206 | TRIM8 | −0.42 | 4.61E−05 | 7.84E−03 | short | JAKSTAT | Univariant |
| | | | | | | signaling | |
| ENSG00000185338 | SOCS1 | 1.60 | 1.50E−06 | 6.89E−04 | short | JAKSTAT | Univariant |
| | | | | | | signaling | |
| ENSG00000197646 | PDCD1LG2 | 1.95 | 1.43E−07 | 1.08E−04 | short | Tcell | Univariant |
| ENSG00000120217 | CD274 | 1.49 | 3.86E−07 | 2.24E−04 | short | Tcell | Univariant |
| ENSG00000186810 | CXCR3 | 0.52 | 1.51E−04 | 1.56E−02 | short | Tcell | Univariant |
| ENSG00000175643 | RMI2 | 1.97 | 1.42E−09 | 2.66E−06 | short | | Univariant |
| ENSG00000238121 | LINC00426 | 0.42 | 2.02E−04 | 1.89E−02 | short | | Multivariant |
| ENSG00000167536 | DHRS13 | −0.76 | 1.58E−05 | 3.74E−03 | short | | Multivariant |
| ENSG00000158714 | SLAMF8 | 1.01 | 2.05E−05 | 4.50E−03 | short | | Multivariant |
| ENSG00000145819 | ARHGAP26 | −0.32 | 6.41E−04 | 3.88E−02 | short | | Multivariant |
| ENSG00000131389 | SLC6A6 | −0.35 | 4.10E−05 | 7.14E−03 | short | | Multivariant |
| ENSG00000165801 | ARHGEF40 | −0.57 | 1.17E−04 | 1.36E−02 | short | | Multivariant |
| ENSG00000197852 | INKA2 | −0.39 | 3.40E−05 | 6.59E−03 | short | | Multivariant |
| ENSG00000239713 | APOBEC3G | 0.36 | 3.58E−05 | 6.77E−03 | short | | Multivariant |
| ENSG00000004468 | CD38 | 0.72 | 1.36E−05 | 3.42E−03 | short | | Univariant |
| ENSG00000120509 | PDZD11 | 0.33 | 6.81E−06 | 2.02E−03 | short | | Univariant |
| ENSG00000133321 | RARRES3 | 0.90 | 1.72E−06 | 7.14E−04 | short | | Univariant |
| ENSG00000178726 | THBD | −0.68 | 4.02E−05 | 7.14E−03 | short | | Univariant |
| ENSG00000181409 | AATK | −1.06 | 6.61E−06 | 2.02E−03 | short | | Univariant |
| ENSG00000224307 | AL161785.1 | −0.59 | 3.83E−05 | 7.06E−03 | short | | Univariant |
| ENSG00000010319 | SEMA3G | 0.00 | 1.00E+00 | 1.00E+00 | short | | Multivariant |
| ENSG00000180871 | CXCR2 | −0.40 | 1.29E−04 | 1.44E−02 | short | | Multivariant |
| ENSG00000131480 | AOC2 | −0.54 | 5.29E−06 | 1.70E−03 | short | | Multivariant |
| ENSG00000154451 | GBP5 | 1.76 | 5.09E−07 | 2.82E−04 | short | | Univariant |
| ENSG00000090104 | RGS1 | 0.00 | 1.00E+00 | 1.00E+00 | short | | Multivariant |
| ENSG00000075426 | FOSL2 | −0.34 | 3.22E−04 | 2.62E−02 | short | | Multivariant |
| ENSG00000102445 | RUBCNL | −0.49 | 1.00E−04 | 1.24E−02 | short | | Univariant |
| ENSG00000130309 | COLGALT1 | −0.30 | 7.93E−05 | 1.09E−02 | short | | Univariant |
| ENSG00000159388 | BTG2 | −0.29 | 5.52E−05 | 8.64E−03 | short | | Univariant |
| ENSG00000167987 | VPS37C | −0.35 | 5.28E−05 | 8.50E−03 | short | | Univariant |
| ENSG00000185650 | ZFP36L1 | −0.35 | 9.61E−05 | 1.21E−02 | short | | Univariant |
| ENSG00000198937 | CCDC167 | 0.44 | 1.33E−04 | 1.46E−02 | short | | Univariant |
| ENSG00000211724 | TRBV6-6 | 0.49 | 2.16E−04 | 1.94E−02 | short | | Univariant |
| ENSG00000211727 | TRBV7-6 | 0.63 | 6.93E−05 | 9.94E−03 | short | | Univariant |
| ENSG00000152969 | JAKMIP1 | 0.32 | 1.10E−03 | 5.13E−02 | short | | Multivariant |
| ENSG00000135069 | PSAT1 | 0.48 | 1.33E−04 | 1.46E−02 | short | | Multivariant |
| ENSG00000010030 | ETV7 | 1.10 | 5.46E−05 | 8.64E−03 | short | | Univariant |
| ENSG00000165046 | LETM2 | −0.68 | 1.26E−05 | 3.24E−03 | short | | Univariant |
| ENSG00000167900 | TK1 | 1.10 | 5.17E−06 | 1.70E−03 | short | | Univariant |
| ENSG00000185386 | MAPK11 | 0.54 | 2.14E−05 | 4.57E−03 | short | | Univariant |
| ENSG00000189057 | FAM111B | 0.98 | 9.92E−06 | 2.73E−03 | short | | Univariant |
| ENSG00000266088 | AC004585.1 | 0.76 | 3.25E−05 | 6.45E−03 | short | | Univariant |
| ENSG00000049768 | FOXP3 | 0.83 | 1.07E−05 | 2.88E−03 | short | | Univariant |
| ENSG00000113068 | PFDN1 | 0.29 | 1.40E−04 | 1.48E−02 | short | | Multivariant |
| ENSG00000125810 | CD93 | −0.40 | 1.84E−04 | 1.76E−02 | short | | Univariant |
| ENSG00000139579 | NABP2 | 0.26 | 9.41E−05 | 1.21E−02 | short | | Univariant |
| ENSG00000277443 | MARCKS | −0.37 | 1.60E−04 | 1.63E−02 | short | | Univariant |
| ENSG00000131979 | GCH1 | 0.64 | 3.65E−05 | 6.81E−03 | short | | Univariant |
| ENSG00000136514 | RTP4 | 1.07 | 1.64E−05 | 3.82E−03 | short | | Univariant |
| ENSG00000213186 | TRIM59 | 0.31 | 5.44E−04 | 3.55E−02 | short | | Multivariant |
| ENSG00000023171 | GRAMD1B | 1.61 | 2.75E−08 | 2.62E−05 | short | | Univariant |
| ENSG00000225492 | GBP1P1 | 0.00 | 1.00E+00 | 1.00E+00 | short | | Univariant |
| ENSG00000143067 | ZNF697 | −0.50 | 6.22E−05 | 9.22E−03 | short | | Univariant |
| ENSG00000167257 | RNF214 | 0.36 | 4.09E−05 | 7.14E−03 | short | | Univariant |
| ENSG00000256262 | USP30-AS1 | 0.56 | 6.22E−05 | 9.22E−03 | short | | Univariant |
| ENSG00000268240 | AC123912.2 | −0.37 | 4.58E−04 | 3.28E−02 | short | | Multivariant |
| ENSG00000006468 | ETV1 | 0.00 | 1.00E+00 | 1.00E+00 | short | | Multivariant |
| ENSG00000078596 | ITM2A | 0.45 | 3.02E−04 | 2.51E−02 | short | | Multivariant |
| ENSG00000176102 | CSTF3 | 0.22 | 9.07E−04 | 4.80E−02 | short | | Univariant |
| ENSG00000117228 | GBP1 | 1.50 | 2.02E−06 | 8.04E−04 | short | | Univariant |
| ENSG00000152766 | ANKRD22 | 1.48 | 4.06E−06 | 1.36E−03 | short | | Univariant |
| ENSG00000183347 | GBP6 | 1.59 | 2.90E−07 | 1.84E−04 | short | | Univariant |
| ENSG00000162772 | ATF3 | 1.60 | 1.71E−06 | 7.14E−04 | short | | Univariant |
| ENSG00000134152 | KATNBL1 | −0.18 | 1.58E−02 | 1.76E−01 | short | | Multivariant |
| ENSG00000142684 | ZNF593 | 0.15 | 2.53E−02 | 2.18E−01 | short | | Multivariant |
| ENSG00000146083 | RNF44 | −0.19 | 5.40E−03 | 1.08E−01 | short | | Multivariant |
| ENSG00000214456 | PLIN5 | −0.28 | 1.89E−03 | 6.78E−02 | short | | Multivariant |
| ENSG00000120875 | DUSP4 | 1.02 | 3.28E−05 | 6.45E−03 | short | | Univariant |
| ENSG00000267940 | AC022762.2 | −0.31 | 4.92E−04 | 3.41E−02 | short | | Multivariant |
| ENSG00000079263 | SP140 | 0.44 | 1.27E−04 | 1.43E−02 | short | | Univariant |
| ENSG00000105520 | PLPPR2 | −0.40 | 8.16E−05 | 1.10E−02 | short | | Univariant |
| ENSG00000128284 | APOL3 | 0.40 | 1.04E−04 | 1.26E−02 | short | | Univariant |
| ENSG00000139180 | NDUFA9 | 0.41 | 1.46E−04 | 1.52E−02 | short | | Univariant |
| ENSG00000140848 | CPNE2 | −0.39 | 5.15E−05 | 8.37E−03 | short | | Univariant |
| ENSG00000163421 | PROK2 | −0.54 | 2.15E−04 | 1.94E−02 | short | | Univariant |
| ENSG00000164687 | FABP5 | 0.51 | 9.87E−05 | 1.23E−02 | short | | Univariant |
| ENSG00000168995 | SIGLEC7 | −0.35 | 8.79E−05 | 1.17E−02 | short | | Univariant |
| ENSG00000143891 | GALM | 0.48 | 1.44E−04 | 1.52E−02 | short | | Univariant |
| ENSG00000124191 | TOX2 | 0.44 | 8.13E−04 | 4.52E−02 | short | | Univariant |
| ENSG00000154640 | BTG3 | 0.37 | 1.61E−04 | 1.64E−02 | short | | Univariant |
| ENSG00000136982 | DSCC1 | 0.00 | 1.00E+00 | 1.00E+00 | | CellCycle | Univariant |
| ENSG00000142945 | KIF2C | 0.30 | 2.44E−03 | 7.65E−02 | | CellCycle | Univariant |
| ENSG00000165304 | MELK | 0.00 | 1.00E+00 | 1.00E+00 | | CellCycle | Univariant |
| ENSG00000163808 | KIF15 | 0.19 | 8.91E−03 | 1.34E−01 | | CellCycle | Univariant |
| ENSG00000198901 | PRC1 | 0.13 | 4.24E−02 | 2.79E−01 | | CellCycle | Univariant |
| ENSG00000151725 | CENPU | 0.61 | 2.19E−05 | 4.61E−03 | | CellCycle | Univariant |
| ENSG00000075218 | GTSE1 | 0.26 | 3.56E−03 | 8.98E−02 | | CellCycle | Univariant |
| ENSG00000101057 | MYBL2 | 0.77 | 5.69E−05 | 8.78E−03 | | CellCycle | Univariant |
| ENSG00000119969 | HELLS | 0.23 | 4.78E−03 | 1.03E−01 | | CellCycle | Univariant |
| ENSG00000139618 | BRCA2 | 0.83 | 1.71E−04 | 1.69E−02 | | CellCycle | Univariant |
| ENSG00000165480 | SKA3 | 0.00 | 1.00E+00 | 1.00E+00 | | CellCycle | Univariant |
| ENSG00000065328 | MCM10 | 0.00 | 1.00E+00 | 1.00E+00 | | CellCycle | Univariant |
| ENSG00000184661 | CDCA2 | 0.00 | 1.00E+00 | 1.00E+00 | | CellCycle | Univariant |
| ENSG00000084764 | MAPRE3 | −0.41 | 7.33E−05 | 1.04E−02 | | CellCycle | Univariant |
| ENSG00000077152 | UBE2T | 0.66 | 9.54E−05 | 1.21E−02 | | CellCycle | Univariant |
| ENSG00000101003 | GINS1 | 0.24 | 3.92E−03 | 9.34E−02 | | CellCycle | Univariant |
| ENSG00000134690 | CDCA8 | 0.45 | 1.19E−04 | 1.38E−02 | | CellCycle | Univariant |
| ENSG00000147889 | CDKN2A | 0.00 | 1.00E+00 | 1.00E+00 | | CellCycle | Univariant |
| ENSG00000175305 | CCNE2 | 0.67 | 1.30E−04 | 1.44E−02 | | CellCycle | Univariant |
| ENSG00000196230 | TUBB | 0.16 | 1.47E−02 | 1.69E−01 | | CellCycle | Multivariant |
| ENSG00000164045 | CDC25A | 0.00 | 1.00E+00 | 1.00E+00 | | CellCycle | Univariant |
| ENSG00000024526 | DEPDC1 | 0.00 | 1.00E+00 | 1.00E+00 | | CellCycle | Univariant |
| ENSG00000129173 | E2F8 | 0.00 | 1.00E+00 | 1.00E+00 | | CellCycle | Univariant |
| ENSG00000138778 | CENPE | 0.34 | 1.89E−03 | 6.78E−02 | | CellCycle | Univariant |
| ENSG00000135083 | CCNJL | −0.36 | 1.03E−03 | 5.05E−02 | | CellCycle | Univariant |
| ENSG00000073111 | MCM2 | 0.40 | 2.80E−04 | 2.38E−02 | | CellCycle | Univariant |
| ENSG00000167513 | CDT1 | 0.50 | 2.98E−04 | 2.49E−02 | | CellCycle | Univariant |
| ENSG00000168496 | FEN1 | 0.30 | 7.40E−04 | 4.29E−02 | | CellCycle | Univariant |
| ENSG00000117724 | CENPF | 0.28 | 3.32E−03 | 8.69E−02 | | CellCycle | Univariant |
| ENSG00000143476 | DTL | 0.29 | 2.83E−03 | 8.16E−02 | | CellCycle | Univariant |
| ENSG00000100479 | POLE2 | 0.31 | 2.53E−03 | 7.75E−02 | | CellCycle | Univariant |
| ENSG00000100526 | CDKN3 | 0.00 | 1.00E+00 | 1.00E+00 | | CellCycle | Univariant |
| ENSG00000149554 | CHEK1 | 0.14 | 2.71E−02 | 2.24E−01 | | CellCycle | Univariant |
| ENSG00000178999 | AURKB | 0.37 | 9.88E−04 | 5.01E−02 | | CellCycle | Univariant |
| ENSG00000028116 | VRK2 | 0.31 | 5.09E−04 | 3.48E−02 | | CellCycle | Univariant |
| ENSG00000119397 | CNTRL | 0.26 | 5.18E−04 | 3.53E−02 | | CellCycle | Univariant |
| ENSG00000141551 | CSNK1D | −0.32 | 1.70E−04 | 1.69E−02 | | CellCycle | Univariant |
| ENSG00000161057 | PSMC2 | 0.29 | 3.03E−04 | 2.51E−02 | | CellCycle | Univariant |
| ENSG00000168078 | PBK | 0.00 | 1.00E+00 | 1.00E+00 | | CellCycle | Univariant |
| ENSG00000071539 | TRIP13 | 0.32 | 1.56E−03 | 6.15E−02 | | CellCycle | Univariant |
| ENSG00000080986 | NDC80 | 0.13 | 4.34E−02 | 2.82E−01 | | CellCycle | Univariant |
| ENSG00000112742 | TTK | 0.33 | 2.04E−03 | 7.03E−02 | | CellCycle | Univariant |
| ENSG00000121621 | KIF18A | 0.27 | 2.97E−03 | 8.34E−02 | | CellCycle | Univariant |
| ENSG00000101447 | FAM83D | 0.37 | 4.73E−04 | 3.36E−02 | | CellCycle | Univariant |
| ENSG00000138182 | KIF20B | 0.34 | 6.67E−04 | 4.00E−02 | | CellCycle | Univariant |
| ENSG00000156802 | ATAD2 | 0.26 | 1.99E−03 | 6.98E−02 | | CellCycle | Univariant |
| ENSG00000276043 | UHRF1 | 0.20 | 8.69E−03 | 1.33E−01 | | CellCycle | Univariant |
| ENSG00000115415 | STAT1 | 0.38 | 7.49E−04 | 4.32E−02 | | JAKSTAT | Univariant |
| | | | | | | signaling | |
| ENSG00000169245 | CXCL10 | 0.96 | 9.24E−05 | 1.21E−02 | | Tcell | Univariant |
| ENSG00000138755 | CXCL9 | 0.00 | 1.00E+00 | 1.00E+00 | | Tcell | Univariant |
| ENSG00000089692 | LAG3 | 0.27 | 3.34E−03 | 8.69E−02 | | Tcell | Univariant |
| ENSG00000134460 | IL2RA | 0.34 | 7.97E−04 | 4.48E−02 | | Tcell | Univariant |
| ENSG00000116824 | CD2 | 0.22 | 5.86E−03 | 1.11E−01 | | Tcell | Multivariant |
| ENSG00000178562 | CD28 | 0.30 | 1.78E−03 | 6.60E−02 | | Tcell | Multivariant |
| ENSG00000011590 | ZBTB32 | 0.00 | 1.00E+00 | 1.00E+00 | | | Univariant |
| ENSG00000070404 | FSTL3 | −0.76 | 5.54E−05 | 8.64E−03 | | | Univariant |
| ENSG00000078081 | LAMP3 | 1.46 | 8.74E−06 | 2.49E−03 | | | Univariant |
| ENSG00000105205 | CLC | 1.17 | 3.53E−05 | 6.76E−03 | | | Univariant |
| ENSG00000134809 | TIMM10 | 0.95 | 1.94E−05 | 4.38E−03 | | | Univariant |
| ENSG00000168062 | BATF2 | 0.97 | 9.51E−05 | 1.21E−02 | | | Univariant |
| ENSG00000168899 | VAMP5 | 0.77 | 4.35E−05 | 7.47E−03 | | | Univariant |
| ENSG00000188820 | CALHM6 | 1.05 | 2.15E−05 | 4.57E−03 | | | Univariant |
| ENSG00000260943 | LINC02555 | 0.99 | 6.96E−05 | 9.94E−03 | | | Univariant |
| ENSG00000149131 | SERPING1 | 1.18 | 3.92E−05 | 7.14E−03 | | | Univariant |
| ENSG00000174944 | P2RY14 | 1.28 | 2.54E−05 | 5.26E−03 | | | Univariant |
| ENSG00000026751 | SLAMF7 | 0.43 | 2.65E−04 | 2.26E−02 | | | Univariant |
| ENSG00000067057 | PFKP | 0.31 | 8.36E−04 | 4.58E−02 | | | Univariant |
| ENSG00000129450 | SIGLEC9 | −0.34 | 2.41E−04 | 2.09E−02 | | | Univariant |
| ENSG00000140511 | HAPLN3 | 0.42 | 4.27E−04 | 3.13E−02 | | | Univariant |
| ENSG00000146094 | DOK3 | −0.37 | 2.39E−04 | 2.09E−02 | | | Univariant |
| ENSG00000204054 | LINC00963 | −0.34 | 3.77E−04 | 2.97E−02 | | | Univariant |
| ENSG00000211714 | TRBV7-3 | 0.49 | 3.62E−04 | 2.87E−02 | | | Univariant |
| ENSG00000181826 | RELL1 | −0.33 | 3.83E−04 | 3.00E−02 | | | Univariant |
| ENSG00000137872 | SEMA6D | 0.00 | 1.00E+00 | 1.00E+00 | | | Univariant |
| ENSG00000155754 | C2CD6 | 0.00 | 1.00E+00 | 1.00E+00 | | | Univariant |
| ENSG00000224843 | LINC00240 | 0.00 | 1.00E+00 | 1.00E+00 | | | Univariant |
| ENSG00000233593 | AL590094.1 | 0.00 | 1.00E+00 | 1.00E+00 | | | Univariant |
| ENSG00000129353 | SLC44A2 | −0.27 | 2.32E−04 | 2.04E−02 | | | Multivariant |
| ENSG00000146828 | SLC12A9 | −0.35 | 1.81E−04 | 1.76E−02 | | | Multivariant |
| ENSG00000169180 | XPO6 | −0.32 | 5.56E−04 | 3.60E−02 | | | Multivariant |
| ENSG00000086205 | FOLH1 | 0.00 | 1.00E+00 | 1.00E+00 | | | Univariant |
| ENSG00000132535 | DLG4 | −0.42 | 1.40E−04 | 1.48E−02 | | | Univariant |
| ENSG00000135451 | TROAP | 0.63 | 2.90E−04 | 2.43E−02 | | | Univariant |
| ENSG00000171621 | SPSB1 | 0.18 | 1.52E−02 | 1.72E−01 | | | Univariant |
| ENSG00000172731 | LRRC20 | 0.38 | 1.23E−04 | 1.40E−02 | | | Univariant |
| ENSG00000228363 | 0 | 0.51 | 1.86E−04 | 1.77E−02 | | | Univariant |
| ENSG00000237772 | AC092620.1 | −0.55 | 1.59E−04 | 1.63E−02 | | | Univariant |
| ENSG00000272625 | 0 | −0.44 | 1.34E−04 | 1.46E−02 | | | Univariant |
| ENSG00000186567 | CEACAM19 | −0.65 | 7.51E−05 | 1.04E−02 | | | Univariant |
| ENSG00000070476 | ZXDC | −0.17 | 6.18E−03 | 1.14E−01 | | | Multivariant |
| ENSG00000082996 | RNF13 | −0.04 | 4.51E−01 | 7.52E−01 | | | Multivariant |
| ENSG00000180228 | PRKRA | 0.03 | 5.10E−01 | 7.88E−01 | | | Multivariant |
| ENSG00000181036 | FCRL6 | 0.06 | 2.39E−01 | 5.84E−01 | | | Multivariant |
| ENSG00000239697 | TNFSF12 | −0.10 | 8.70E−02 | 3.83E−01 | | | Multivariant |
| ENSG00000285756 | BX890604.2 | 0.02 | 6.85E−01 | 8.74E−01 | | | Multivariant |
| ENSG00000248769 | AC139495.2 | −1.39 | 5.63E−06 | 1.78E−03 | | | Univariant |
| ENSG00000100336 | APOL4 | 0.23 | 5.10E−03 | 1.06E−01 | | | Univariant |
| ENSG00000134873 | CLDN10 | −0.93 | 1.98E−05 | 4.40E−03 | | | Univariant |
| ENSG00000164112 | TMEM155 | 0.00 | 1.00E+00 | 1.00E+00 | | | Univariant |
| ENSG00000164626 | KCNK5 | 0.00 | 1.00E+00 | 1.00E+00 | | | Univariant |
| ENSG00000177602 | HASPIN | 0.00 | 1.00E+00 | 1.00E+00 | | | Univariant |
| ENSG00000126603 | GLIS2 | 0.00 | 1.00E+00 | 1.00E+00 | | | Univariant |
| ENSG00000115594 | IL1R1 | −0.19 | 1.12E−02 | 1.47E−01 | | | Multivariant |
| ENSG00000113749 | HRH2 | −0.47 | 1.70E−04 | 1.69E−02 | | | Univariant |
| ENSG00000119535 | CSF3R | −0.38 | 9.06E−05 | 1.19E−02 | | | Univariant |
| ENSG00000127948 | POR | −0.61 | 1.01E−04 | 1.24E−02 | | | Univariant |
| ENSG00000162551 | ALPL | −0.71 | 1.99E−04 | 1.88E−02 | | | Univariant |
| ENSG00000166825 | ANPEP | −0.43 | 1.38E−04 | 1.48E−02 | | | Univariant |
| ENSG00000175274 | TP53111 | −0.58 | 8.00E−05 | 1.09E−02 | | | Univariant |
| ENSG00000211750 | TRBV24-1 | 0.30 | 2.13E−03 | 7.14E−02 | | | Univariant |
| ENSG00000142552 | RCN3 | −0.47 | 4.78E−05 | 7.86E−03 | | | Univariant |
| ENSG00000097021 | ACOT7 | 0.35 | 6.05E−04 | 3.75E−02 | | | Univariant |
| ENSG00000141013 | GAS8 | 0.37 | 7.69E−04 | 4.39E−02 | | | Univariant |
| ENSG00000167566 | NCKAP5L | −0.32 | 7.66E−04 | 4.39E−02 | | | Univariant |
| ENSG00000206028 | Z99774.1 | 0.44 | 4.31E−04 | 3.15E−02 | | | Univariant |
| ENSG00000167535 | CACNB3 | −0.63 | 6.38E−05 | 9.36E−03 | | | Univariant |
| ENSG00000211655 | IGLV1-36 | 0.00 | 1.00E+00 | 1.00E+00 | | | Univariant |
| ENSG00000267416 | AC025048.4 | 0.00 | 1.00E+00 | 1.00E+00 | | | Univariant |
| ENSG00000284681 | 0 | 0.00 | 1.00E+00 | 1.00E+00 | | | Univariant |
| ENSG00000108387 | 38231 | 0.09 | 6.40E−02 | 3.37E−01 | | | Univariant |
| ENSG00000156639 | ZFAND3 | −0.31 | 6.88E−04 | 4.06E−02 | | | Univariant |
| ENSG00000197405 | C5AR1 | −0.32 | 4.10E−04 | 3.07E−02 | | | Univariant |
| ENSG00000008283 | CYB561 | 0.36 | 3.54E−04 | 2.82E−02 | | | Univariant |
| ENSG00000073150 | PANX2 | −0.36 | 4.56E−04 | 3.28E−02 | | | Univariant |
| ENSG00000100034 | PPM1F | −0.30 | 4.82E−04 | 3.38E−02 | | | Univariant |
| ENSG00000105656 | ELL | −0.32 | 2.58E−04 | 2.22E−02 | | | Univariant |
| ENSG00000181220 | ZNF746 | −0.31 | 4.05E−04 | 3.07E−02 | | | Univariant |
| ENSG00000018280 | SLC11A1 | −0.36 | 6.87E−04 | 4.06E−02 | | | Univariant |
| ENSG00000051009 | FAM160A2 | −0.31 | 1.66E−04 | 1.67E−02 | | | Univariant |
| ENSG00000065427 | KARS | 0.28 | 3.37E−04 | 2.71E−02 | | | Univariant |
| ENSG00000069399 | BCL3 | −0.39 | 5.41E−04 | 3.55E−02 | | | Univariant |
| ENSG00000072952 | MRVI1 | −0.41 | 3.11E−04 | 2.54E−02 | | | Univariant |
| ENSG00000079432 | CIC | −0.30 | 8.32E−04 | 4.58E−02 | | | Univariant |
| ENSG00000099308 | MAST3 | −0.31 | 2.89E−04 | 2.43E−02 | | | Univariant |
| ENSG00000099331 | MYO9B | −0.26 | 8.54E−04 | 4.61E−02 | | | Univariant |
| ENSG00000100266 | PACSIN2 | −0.33 | 3.98E−04 | 3.06E−02 | | | Univariant |
| ENSG00000100284 | TOM1 | −0.32 | 4.14E−04 | 3.07E−02 | | | Univariant |
| ENSG00000107020 | PLGRKT | 0.27 | 7.51E−04 | 4.32E−02 | | | Univariant |
| ENSG00000108175 | ZMIZ1 | −0.29 | 9.10E−04 | 4.80E−02 | | | Univariant |
| ENSG00000110395 | CBL | −0.28 | 5.68E−04 | 3.64E−02 | | | Univariant |
| ENSG00000120318 | ARAP3 | −0.35 | 2.01E−04 | 1.89E−02 | | | Univariant |
| ENSG00000120327 | PCDHB14 | 0.00 | 1.00E+00 | 1.00E+00 | | | Univariant |
| ENSG00000127663 | KDM4B | −0.28 | 9.56E−04 | 4.90E−02 | | | Univariant |
| ENSG00000129003 | VPS13C | 0.25 | 5.73E−04 | 3.64E−02 | | | Univariant |
| ENSG00000130382 | MLLT1 | −0.30 | 8.60E−04 | 4.63E−02 | | | Univariant |
| ENSG00000131626 | PPFIA1 | −0.28 | 6.90E−04 | 4.06E−02 | | | Univariant |
| ENSG00000131943 | C19orf12 | 0.28 | 1.20E−03 | 5.32E−02 | | | Univariant |
| ENSG00000132510 | KDM6B | −0.32 | 5.60E−04 | 3.60E−02 | | | Univariant |
| ENSG00000132514 | CLEC10A | 0.31 | 8.42E−04 | 4.58E−02 | | | Univariant |
| ENSG00000133997 | MED6 | 0.21 | 5.46E−04 | 3.55E−02 | | | Univariant |
| ENSG00000134594 | RAB33A | 0.32 | 4.76E−04 | 3.36E−02 | | | Univariant |
| ENSG00000134815 | DHX34 | −0.33 | 4.01E−04 | 3.06E−02 | | | Univariant |
| ENSG00000136045 | PWP1 | 0.28 | 1.01E−04 | 1.24E−02 | | | Univariant |
| ENSG00000136830 | FAM129B | −0.34 | 5.00E−04 | 3.46E−02 | | | Univariant |
| ENSG00000137573 | SULF1 | 0.00 | 1.00E+00 | 1.00E+00 | | | Univariant |
| ENSG00000138061 | CYP1B1 | −0.41 | 5.95E−04 | 3.70E−02 | | | Univariant |
| ENSG00000140995 | DEF8 | −0.28 | 5.44E−04 | 3.55E−02 | | | Univariant |
| ENSG00000142405 | NLRP12 | −0.38 | 1.35E−04 | 1.46E−02 | | | Univariant |
| ENSG00000146826 | C7orf43 | −0.26 | 8.45E−04 | 4.58E−02 | | | Univariant |
| ENSG00000149798 | CDC42EP2 | −0.35 | 4.10E−04 | 3.07E−02 | | | Univariant |
| ENSG00000163464 | CXCR1 | −0.33 | 5.41E−04 | 3.55E−02 | | | Univariant |
| ENSG00000163545 | NUAK2 | −0.28 | 7.38E−04 | 4.29E−02 | | | Univariant |
| ENSG00000163739 | CXCL1 | −0.30 | 8.44E−04 | 4.58E−02 | | | Univariant |
| ENSG00000165806 | CASP7 | 0.30 | 4.23E−04 | 3.12E−02 | | | Univariant |
| ENSG00000166145 | SPINT1 | −0.40 | 4.14E−04 | 3.07E−02 | | | Univariant |
| ENSG00000166987 | MBD6 | −0.30 | 4.38E−04 | 3.19E−02 | | | Univariant |
| ENSG00000168067 | MAP4K2 | −0.30 | 3.86E−04 | 3.00E−02 | | | Univariant |
| ENSG00000170190 | SLC16A5 | −0.33 | 1.08E−04 | 1.30E−02 | | | Univariant |
| ENSG00000171608 | PIK3CD | −0.27 | 8.13E−04 | 4.52E−02 | | | Univariant |
| ENSG00000173535 | TNFRSF10C | −0.34 | 3.87E−04 | 3.00E−02 | | | Univariant |
| ENSG00000176788 | BASP1 | −0.34 | 9.66E−04 | 4.94E−02 | | | Univariant |
| ENSG00000177169 | ULK1 | −0.29 | 9.27E−04 | 4.81E−02 | | | Univariant |
| ENSG00000181444 | ZNF467 | −0.33 | 9.31E−04 | 4.81E−02 | | | Univariant |
| ENSG00000182885 | ADGRG3 | −0.41 | 3.33E−04 | 2.69E−02 | | | Univariant |
| ENSG00000185880 | TRIM69 | 0.32 | 6.31E−04 | 3.85E−02 | | | Univariant |
| ENSG00000185963 | BICD2 | −0.29 | 5.73E−04 | 3.64E−02 | | | Univariant |
| ENSG00000186635 | ARAP1 | −0.32 | 1.14E−04 | 1.33E−02 | | | Univariant |
| ENSG00000196562 | SULF2 | −0.28 | 8.96E−04 | 4.77E−02 | | | Univariant |
| ENSG00000197894 | ADH5 | 0.29 | 3.41E−04 | 2.73E−02 | | | Univariant |
| ENSG00000198673 | FAM19A2 | 0.44 | 6.78E−04 | 4.04E−02 | | | Univariant |
| ENSG00000198792 | TMEM184B | −0.28 | 9.14E−04 | 4.80E−02 | | | Univariant |
| ENSG00000198933 | TBKBP1 | −0.34 | 5.80E−04 | 3.66E−02 | | | Univariant |
| ENSG00000204304 | PBX2 | −0.24 | 7.12E−04 | 4.17E−02 | | | Univariant |
| ENSG00000213876 | RPL7AP64 | −0.29 | 6.66E−04 | 4.00E−02 | | | Univariant |
| ENSG00000280734 | LINC01232 | 0.37 | 5.32E−04 | 3.55E−02 | | | Univariant |
| ENSG00000269711 | AC008763.3 | 0.02 | 6.25E−01 | 8.49E−01 | | | Multivariant |
| ENSG00000246548 | LINC02288 | −0.54 | 2.32E−04 | 2.04E−02 | | | Univariant |
| ENSG00000107614 | TRDMT1 | 0.01 | 9.29E−01 | 9.74E−01 | | | Multivariant |
| ENSG00000106809 | OGN | 0.00 | 1.00E+00 | 1.00E+00 | | | Univariant |
| ENSG00000254602 | AP000662.1 | 0.00 | 1.00E+00 | 1.00E+00 | | | Univariant |
| ENSG00000002549 | LAP3 | 0.71 | 1.21E−04 | 1.39E−02 | | | Univariant |
| ENSG00000125864 | BFSP1 | 0.29 | 1.85E−03 | 6.71E−02 | | | Univariant |
| ENSG00000165409 | TSHR | 0.61 | 3.07E−04 | 2.53E−02 | | | Univariant |
| ENSG00000185499 | MUC1 | 0.70 | 2.28E−04 | 2.03E−02 | | | Univariant |
| ENSG00000188343 | FAM92A | 0.75 | 2.05E−04 | 1.91E−02 | | | Univariant |
| ENSG00000203879 | GDI1 | −0.23 | 7.73E−04 | 4.40E−02 | | | Univariant |
| ENSG00000144580 | CNOT9 | 0.23 | 8.15E−04 | 4.52E−02 | | | Univariant |
| ENSG00000136161 | RCBTB2 | −0.23 | 9.19E−04 | 4.81E−02 | | | Univariant |
| ENSG00000060656 | PTPRU | 0.00 | 1.00E+00 | 1.00E+00 | | | Univariant |
| ENSG00000111879 | FAM184A | 1.22 | 6.77E−06 | 2.02E−03 | | | Univariant |
| ENSG00000113645 | WWC1 | 1.04 | 6.43E−05 | 9.36E−03 | | | Univariant |
| ENSG00000143816 | WNT9A | 0.00 | 1.00E+00 | 1.00E+00 | | | Univariant |
| ENSG00000157064 | NMNAT2 | 0.00 | 1.00E+00 | 1.00E+00 | | | Univariant |
| ENSG00000171246 | NPTX1 | 0.00 | 1.00E+00 | 1.00E+00 | | | Univariant |
| ENSG00000198785 | GRIN3A | 0.84 | 1.12E−04 | 1.33E−02 | | | Univariant |
| ENSG00000104381 | GDAP1 | 0.37 | 2.13E−04 | 1.94E−02 | | | Univariant |
| ENSG00000221926 | TRIM16 | 0.31 | 5.89E−04 | 3.68E−02 | | | Univariant |
| ENSG00000087903 | RFX2 | −0.37 | 8.08E−04 | 4.52E−02 | | | Univariant |
| ENSG00000105072 | C19orf44 | −0.50 | 5.36E−04 | 3.55E−02 | | | Univariant |
| ENSG00000180758 | GPR157 | −0.34 | 6.10E−04 | 3.76E−02 | | | Univariant |
| ENSG00000249673 | NOP14-AS1 | 0.29 | 9.32E−04 | 4.81E−02 | | | Univariant |
| ENSG00000258102 | MAP1LC3B2 | −0.42 | 5.37E−04 | 3.55E−02 | | | Univariant |
| ENSG00000019991 | HGF | −0.37 | 5.38E−04 | 3.55E−02 | | | Univariant |
| ENSG00000100523 | DDHD1 | 0.34 | 5.43E−04 | 3.55E−02 | | | Univariant |
| ENSG00000108679 | LGALS3BP | 0.43 | 6.78E−04 | 4.04E−02 | | | Univariant |
| ENSG00000114853 | ZBTB47 | −0.32 | 3.90E−04 | 3.01E−02 | | | Univariant |
| ENSG00000120254 | MTHFD1L | 0.30 | 1.46E−03 | 5.93E−02 | | | Univariant |
| ENSG00000128394 | APOBEC3F | 0.28 | 1.18E−03 | 5.29E−02 | | | Univariant |
| ENSG00000138119 | MYOF | 0.40 | 6.21E−04 | 3.81E−02 | | | Univariant |
| ENSG00000166398 | KIAA0355 | −0.26 | 5.03E−04 | 3.46E−02 | | | Univariant |
| ENSG00000175556 | LONRF3 | −0.38 | 2.11E−04 | 1.94E−02 | | | Univariant |
| ENSG00000187474 | FPR3 | −0.42 | 5.81E−04 | 3.66E−02 | | | Univariant |
| ENSG00000233901 | LINC01503 | −0.44 | 1.79E−04 | 1.76E−02 | | | Univariant |
| ENSG00000241978 | AKAP2 | 0.41 | 5.84E−04 | 3.67E−02 | | | Univariant |
| ENSG00000253522 | MIR3142HG | 0.33 | 9.32E−04 | 4.81E−02 | | | Univariant |
| ENSG00000259342 | AC025580.1 | −0.31 | 8.20E−04 | 4.53E−02 | | | Univariant |
| ENSG00000264910 | RN7SL525P | −0.41 | 5.29E−04 | 3.55E−02 | | | Univariant |
| ENSG00000276017 | AC007325.1 | −0.46 | 8.83E−04 | 4.72E−02 | | | Univariant |
| ENSG00000279095 | AC243964.3 | −0.30 | 6.14E−04 | 3.78E−02 | | | Univariant |
| ENSG00000279447 | AL118508.4 | −0.44 | 4.80E−04 | 3.38E−02 | | | Univariant |
| ENSG00000129657 | SEC14L1 | −0.26 | 1.59E−03 | 6.24E−02 | | | Multivariant |
| ENSG00000137642 | SORL1 | −0.23 | 3.80E−03 | 9.20E−02 | | | Multivariant |
| ENSG00000147443 | DOK2 | 0.02 | 6.65E−01 | 8.65E−01 | | | Multivariant |
| ENSG00000172215 | CXCR6 | 0.31 | 1.90E−03 | 6.78E−02 | | | Multivariant |
| ENSG00000196083 | IL1RAP | −0.20 | 9.35E−03 | 1.37E−01 | | | Multivariant |
| ENSG00000148734 | NPFFR1 | −0.06 | 3.05E−01 | 6.44E−01 | | | Multivariant |
| ENSG00000214336 | FOXI3 | 0.00 | 1.00E+00 | 1.00E+00 | | | Multivariant |
| ENSG00000273599 | AL731571.1 | 0.00 | 1.00E+00 | 1.00E+00 | | | Multivariant |
| ENSG00000154262 | ABCA6 | −0.71 | 2.56E−04 | 2.22E−02 | | | Univariant |
| ENSG00000133800 | LYVE1 | −0.33 | 1.68E−03 | 6.40E−02 | | | Univariant |
| ENSG00000284946 | AC068831.7 | 0.46 | 1.10E−04 | 1.31E−02 | | | Univariant |
| ENSG00000162069 | BICDL2 | −0.45 | 9.05E−04 | 4.80E−02 | | | Univariant |
| ENSG00000163083 | INHBB | −0.46 | 7.93E−04 | 4.48E−02 | | | Univariant |
| ENSG00000166263 | STXBP4 | 0.42 | 9.51E−04 | 4.90E−02 | | | Univariant |
| ENSG00000175894 | TSPEAR | −0.44 | 8.70E−04 | 4.67E−02 | | | Univariant |
| ENSG00000188487 | INSC | −0.60 | 4.46E−04 | 3.23E−02 | | | Univariant |
| ENSG00000197702 | PARVA | −0.49 | 7.93E−04 | 4.48E−02 | | | Univariant |

In one aspect, the invention relates to method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising at least one gene selected among:
the Ribosomal biogenesis genes cluster,
the TCR signaling genes cluster,
the Cilia genes cluster,
the Interferon pathway genes cluster,
or a combination of one or more thereof,
   wherein differential transcription and/or expression and/or activity level of the gene panel, in the biological sample, relative to the level of corresponding transcription and/or expression and/or activity level of the gene panel determined previously, is predictive of the patient's response to said treatment.

In one aspect, the gene panel comprises at least one gene among those listed in Table 1.

In one aspect, the gene panel comprises
at least one, at least 2, at least 3, at least 4, at least 5, at least 6, at least 7, at least 8, at least 9, at list 10, at least 11, at least 12, at least 13, at least 14, at least 15, at least 16, at least 17, at least 18, at least 19, at least 20, at least 21, at least 22, at least, 23, at least 24, at least 25 genes selected among the “best 25” gene list in Table 1 and/or
at least one, at least 5, at least 8, at least 10, at least 20, at least 30, at least 40, at least 50, at least 60, at least 70, at least 80, at least 90, at least 100, at least 110, at least 119 genes selected among the “short” gene list in Table 1.

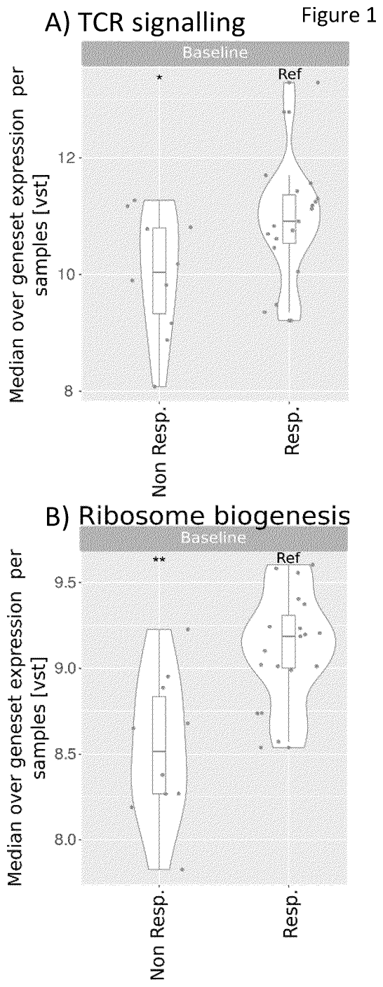

In one aspect of the invention and referring in more details to the examples and to FIG. 1, the 7 genes included in the TCR signaling cluster (CD8B, CD8A, CHI3L2, GZMH, IL23, JAKMIP1 and MIAT), CB+ patients (Resp) exhibit a significant higher expression of this set of genes, in comparison to CB− patients (Non Resp). In parallel, 6 genes (TSR2, GRWD1, RRS1, GLTSCR2, WBSCR22, NOB1) related to Ribosomal biogenesis pathway, exhibited a higher expression ratio in CB+ patients (Resp) versus CB− patients (Non Resp).

Where at least one gene is selected among the TCR signaling genes cluster, the at least one gene is selected among the group of genes comprising, or consisting of, CD8B, CD8A, CHI3L2, GZMH, IL23, JAKMIP1 and MIAT.

Where at least one gene is selected among the Ribosomal biogenesis genes cluster, the at least one gene is selected among the group of genes comprising, or consisting of, TSR2, GRWD1, RRS1, GLTSCR2, WBSCR22, and NOB1.

Where at least one gene is selected among the Cilia genes cluster, the at least one gene is selected among the group of genes comprising, or consisting of, EFCAB2, ENKUR, IQCA1, and IQCD.

Where at least one gene is selected among the Interferon genes cluster, the at least one gene is selected among the group of genes comprising, or consisting of, UNC93B1, APOBEC3B, MLKL, USP15, IFIT2, IRF7, BATF2, PARP9, SAMD9, PLSCR1, DTX3L, ZC3HAV1, IFIT5, TDRD7, and LAMP3. Preferably, the at least one gene is selected among the group of genes comprising, or consisting of, UNC93B1, APOBEC3B, MLKL, USP15, IFIT2, IRF7, and BATF2.

In one aspect, the differential transcription and/or expression and/or activity level of the gene panel corresponds to a differential expression of the transcripts of the one or more genes of the panel.

In one aspect, the differential transcription and/or expression and/or activity level of the gene panel corresponds to a downregulated of said one or more genes of the panel.

Preferably, the downregulated differential transcription and/or expression and/or activity of said gene panel corresponds to a decrease equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding transcription and/or expression and/or activity level of the gene panel determined previously. Examples of gene panels showing downregulated differential transcription and/or expression and/or activity are selected from the group comprising the Interferon pathway genes cluster and the Cilia genes cluster.

In one aspect, the differential transcription and/or expression and/or activity level of the gene panel corresponds to an upregulated expression of said one or more genes of the panel.

Preferably, the upregulated differential transcription and/or expression and/or activity of said gene panel corresponds to an increase equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding transcription and/or expression and/or activity level of the gene panel determined previously. Examples of gene panels showing upregulated differential transcription and/or expression and/or activity are selected from the group comprising the Ribosomal biogenesis genes cluster and the TCR signaling genes cluster.

In case the patient having a predetermined disease (such as cancer) is predicted to respond to said treatment, the treatment is started, or if already started the treatment is continued.

In another or alternative aspect, if the patient having a predetermined disease is predicted not to respond to said treatment, the method further comprises a step of adapting the treatment.

Adapting the treatment comprises not administering the envisioned treatment or inhibitor and/or further administering a combination therapy, and/or adapting the dose, amount and/or regimen, e.g. the treatment, such as e.g. the ICB treatment described herein.

The term “combination therapy” refers to treatments in which an ICB treatment described herein, and another cancer therapy selected from the group comprising immunotherapy, hormonotherapy, targeted therapy, cell therapy, chemotherapy and radiotherapy, administered to a patient in a coordinated manner, over an overlapping period of time.

Usually, the level of corresponding transcription and/or expression and/or activity level of the gene panel determined previously corresponds to a control sample, a reference sample, a group of reference samples, or a reference value.

In one aspect, the control sample has been determined in a biological sample of the same patient before starting the ICBT (i.e. control sample or baseline).

Preferably, the determination has been done about at least 1 month before, about at least 1 week before, about at least one day before, about at least 1 hour, about at least 1 minute before starting the treatment. Alternatively, the biological sample has been collected before starting the treatment, but the determination is done after starting the treatment.

In one aspect, the reference sample or group of reference samples has been determined in a biological sample of either subjects with clinical benefit (CB+) or subjects without clinical benefit (CB−).

In one aspect, the reference value refers to a value (e.g. an absolute value) that has been determined in a biological sample of the same patient, another subject or group of subjects (e.g. CB+ or CB−), using a method described herein.

In another related aspect, the invention relates to a method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising at least one gene selected among:
the Cell Cycle genes cluster,
the Jak/Stat Signaling pathway genes cluster, and
the T cell/Immune tolerance regulation genes cluster,
or a combination of one or more thereof,
wherein differential transcription and/or expression and/or activity level of the gene panel, in the biological sample, relative to the level of corresponding transcription and/or expression and/or activity level of the gene panel determined previously in a reference sample, is predicting that the patient is responsive to said treatment.

In one aspect, the gene panel comprises at least one gene among those listed in Table 2.

In one aspect, the gene panel comprises
at least one, at least 2, at least 3, at least 4, at least 5, at least 6, at least 7, at least 8, at least 9, at list 10, at least 11, at least 12, at least 13, at least 14, at least 15, at least 16, at least 17, at least 18, at least 19, at least 20, at least 21, at least 22, at least, 23, at least 24, at least 25 genes selected among the “best 25” gene list in Table 2 and/or
at least one, at least 5, at least 8, at least 10, at least 20, at least 30, at least 40, at least 50, at least 60, at least 70, at least 80, at least 90, at least 100, at least 110, at least 120, at least 130, at least 141 genes selected among the “short” gene list in Table 2.

Where at least one gene is selected among the Cell Cycle genes cluster, the at least one gene is selected among the group of genes comprising, or consisting of, CDCA7, CDC20, PTTG1, CCNB2, RRM2, NCAPG, TYMS, TPX2, MKI67, KIF11, CCNA2, EZH2, CCNB1, DLGAP5, GMNN, ASPM, RAD51, TOP2A, BUB1, and NCAPH. In one aspect, the gene panel comprises, or consists of, the gene set CDCA7, CDC20, CCNB2, TPX2, and MKI67.

Where at least one gene is selected among the Jak/Stat Signaling pathway genes cluster, the at least one gene is selected among the group of genes comprising, or consisting of, STAT1, SOCS1, STAT6, and TRIM8. In one aspect, the gene panel comprises, or consists of, the gene set STAT1 and SOCS1.

Where at least one gene is selected among the T cell/Immune tolerance regulation genes cluster, the at least one gene is selected among the group of genes comprising, or consisting of, PDCD1, IDO1, CTLA4, CCR4, AOC3, LAG3, CXCR3, CD274, CXCL10, and CXCL9. In one aspect, the gene panel comprises, or consists of, the gene set PDCD1, IDO1, CTLA4, and LAG3.

Preferably, the disease is cancer as disclosed herein. More preferably, the cancer is melanoma, even more preferably metastatic melanoma.

In one aspect, the metastatic melanoma is a melanoma bearing a BRAF gene mutation (e.g. BRAF V600e gene mutation).

In one aspect, the differential transcription and/or expression and/or activity level of the gene panel corresponds to a differential expression of the transcripts of the one or more genes of the panel.

Preferably, the differential transcription and/or expression and/or activity level of the gene panel corresponds to a downregulated or upregulated expression of said one or more genes of the panel.

Preferably, the downregulated differential transcription and/or expression and/or activity of said gene panel corresponds to a decrease equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding transcription and/or expression and/or activity level of the gene panel determined previously. Examples of gene panels showing downregulated differential transcription and/or expression and/or activity are selected from the group comprising at least one gene selected from TLE3, AOC3, AOC2, AATK, LETM2, MAK, DHRS13, INKA2, AL161785.1, THBD, I7NKA2, AL161785.1, THBD, SLC6A6, TRIM8, CPNE2, VPS37C, BTG2, and ZNF697.

Preferably also, the upregulated differential transcription and/or expression and/or activity of said gene panel corresponds to an increase equal or superior to about 5%, preferably equal or superior to about 20%, more preferably equal or superior to about 40%, most preferably equal or superior to about 60%, more preferably equal or superior to about 500%, even more preferably equal or superior to about 1000%, in particular equal or superior to about 5000% when compared to the level of corresponding transcription and/or expression and/or activity level of the gene panel determined previously. Examples of gene panels showing upregulated differential transcription and/or expression and/or activity are selected from the group comprising Cell Cycle genes and Jak/Stat Signaling pathway genes.

In case the patient having a predetermined disease is determined as responsive (i.e. CB+) to said treatment, the treatment is continued.

In another or alternative aspect, if the patient having a predetermined disease is determined as non-responsive to said treatment (i.e. CB−), the method further comprises a step of adapting the treatment.

Adapting the treatment comprises changing the treatment for another treatment or adapting the dose and/or regimen of the treatment, such as e.g. the ICB treatment described herein.

Adapting the treatment comprises administering a combination therapy, and/or adapting the dose and/or regimen of the treatment based on ICBT as disclosed herein.

Usually, the level of transcription and/or expression and/or activity of the gene panel is detected in the biological sample between about 1 to about 16 weeks, about 2 to about 14 weeks, about 2 to about 12 weeks, about 2 to about 10 weeks, after the treatment based on ICBT has started.

In one aspect, the level of transcription and/or expression and/or activity of the gene panel is detected in the biological sample before the start of the treatment.

In one aspect, the samples are collected at the end of 2 cycles (t=6 weeks) or 4 cycles (t=12 weeks) of ICBT based treatment.

Usually, the level of corresponding transcription and/or expression and/or activity level of the gene panel detected are compared to the of corresponding transcription and/or expression and/or activity level of the gene panel determined previously and corresponding to a control sample, a reference sample, a group of reference samples, or a reference value.

In another related aspect, the invention relates to a computer-implemented method for implementing a method for predicting if a patient having a predetermined disease will respond to a treatment based on immune checkpoint blockade therapy or treatment (ICBT) of the invention, said computer-implemented method comprising
i) scoring the level of transcription and/or expression and/or activity of a gene panel in the biological sample of the patient,
ii) comparing the determined score to the score of the gene panel determined previously, whereby difference in the score, in the biological sample, relative to the score of the gene panel determined previously, is predictive of the patient's response to said treatment.

In another related aspect, the invention relates to a computer-implemented method for implementing a method for determining if a patient having a predetermined disease is responsive to a treatment based on immune checkpoint blockade therapy or treatment (ICBT) of any one of the invention, said computer-implemented method comprising
i) scoring the level of transcription and/or expression and/or activity of a gene panel in the biological sample of the patient,
ii) comparing the determined score of the gene panel determined previously, whereby difference in the score, in the biological sample, relative to the score of the gene panel determined previously, is indicative of whether the patient is responsive or not to said treatment.

As used herein, scoring the level of transcription and/or expression and/or activity of a gene panel means transforming the gene expression level of several genes of a panel into a score, with one of the methods described above.

In one aspect, the computer-implemented methods described above involve the use of a computer, computer network or other programmable apparatus.

The present invention also contemplates the use of a gene panel comprising at least one gene selected among
the Cell Cycle genes cluster,
the Jak/Stat Signaling pathway genes cluster, and
the T cell/Immune tolerance genes cluster,
or a combination of one or more thereof, for predicting if a patient having a predetermined disease will respond to a treatment based on immune checkpoint blockade therapy or treatment (ICBT).

The present invention also contemplates the use of a gene panel comprising at least one gene selected among
the Cell Cycle genes cluster,
the Jak/Stat Signaling pathway genes cluster, and
the T cell/Immune tolerance genes cluster,
or a combination of one or more thereof, for determining if a patient having a predetermined disease is responsive to a treatment based on immune checkpoint blockade therapy or treatment (ICBT).

Also encompassed in the present invention is a kit for performing a method of the invention, said kit comprising
a) means and/or reagents for determining the level of transcription and/or expression and/or activity of said gene panel in a biological sample from said patient as described herein, and
b) instructions for use.

The reagents may be packaged in separate containers. The kit may further comprise one or more control reference samples and reagents for performing a method of the invention.

In certain aspects, the kit contains at least one probe to which a particular polynucleotide molecule specifically hybridizes as described herein.

In one aspect, the kit comprises at least one reagent for measuring the level of transcription and/or expression and/or activity of a gene panel.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing a cardiac pathology or monitoring stem cell therapy or regenerative medical treatments.

The kit can also contain a microarray comprising a support or surface with an ordered array of binding (e.g., hybridization) sites or “probes” each representing one of the genes described herein.

Also encompassed in the present invention are methods of treatment of cancer, preferably melanoma, more preferably metastatic melanoma.

In one aspect, the invention discloses a method of treatment of cancer, comprising
i) detecting in a biological sample obtained from said patient the level of transcription and/or expression and/or activity of a gene panel of the invention,
ii) and treating the patient based upon whether a differential transcription and/or expression and/or activity level of said gene panel, in the biological sample, relative to the level of corresponding transcription and/or expression and/or activity level of the gene panel determined previously, is predictive of the patient's response to said treatment.

In case the patient having a predetermined disease (such as cancer) is predicted to respond to said treatment, the envisioned treatment (i.e. ICB treatment) is started.

In another or alternative aspect, if the patient having a predetermined disease is predicted not to respond to said treatment, the method further comprises a step of adapting the treatment.

Adapting the treatment comprises not administering the envisioned treatment or inhibitor and/or adapting the dose, amount or regimen of, e.g. the treatment, such as e.g. the ICB treatment described herein.

In one aspect, the invention discloses a method of treatment of cancer, comprising
i) detecting in a biological sample obtained from said patient the level of transcription and/or expression and/or activity of a gene panel of the invention,
ii) and treating the patient based upon whether a differential transcription and/or expression and/or activity level of said gene panel, in the biological sample, relative to the level of corresponding transcription and/or expression and/or activity level of the gene panel determined previously, is determining that the patient is responsive or not to said treatment.

In case the patient having a predetermined disease is determined as responsive to said treatment, the treatment is continued.

In another or alternative aspect, if the patient having a predetermined disease is determined as non-responsive to said treatment, the method further comprises a step of adapting the treatment.

The step of adapting the treatment comprises changing the treatment for another treatment or adapting the dose and/or regimen of the treatment.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein. Various references are cited throughout this Specification, each of which is incorporated herein by reference in its entirety. The foregoing description will be more fully understood with reference to the following Examples.

### EXAMPLES

### Materials and Methods

### Patient Cohort and Study Design

The present retrospective study included 29 patients with metastatic melanoma from a phase II trial. In this study, patients with BRAF+ (BRAFV600E/K mutation-positive), high LDH (elevated serum lactate dehydrogenase) late-stage (IV) melanoma were treated with immune checkpoint inhibitors (ICI). In this arm, an anti-PD-1/CTLA-4 therapy as first-line treatment was administered, at the Radboud University Medical Center, from 2017 to 2021. Patients were treated with ipilimumab 3 mg/kg and nivolumab 1 mg/kg every 3 weeks, during 4 cycles. Patient objective response was assessed according to RECIST 1.1 criteria at 12 weeks. Clinical benefit (CB+) was considered in patients with clinical progression-free survival (PFS) higher than 6 months exhibiting both complete and/or partial response. Conversely, patients with PFS lower than six months, showing stable disease or progression in disease were classified as having no clinical benefit (CB−).

Simultaneously, whole blood was drawn for predictive and retrospective biomarker studies. Patient cohort characteristics are described in Table 3.

TABLE 3
Patient cohort composition.
General characteristics

| | |
|---|---|
| Total of patients, n (%) | 29 (100) |
| Age, median (range) years | 65 (28-78) |
| Gender, n (%) | |
| Male | 20 (68.9) |
| Female | 9 (31.0) |
| Location of metastases, n (%) | |
| Liver metastases | 13 (44.8) |
| Other metastases | 16 (55.2) |

Treatment with ipilimumab and nivolumab (4 cycles)

| | |
|---|---|
| Patient clinical outcome at baseline, n (%) | 29 (100) |
| PFS <6 months | 10 (34.5) |
| Stable disease | 1 (10.0) |
| Progressive disease | 9 (90.0) |
| PFS ≥6 months | 19 (65.5) |
| Complete response | 3 (15.7) |
| Partial response | 16 (84.2) |
| Patient clinical outcome on treatment, n (%) | 24 (100) |
| at 6 weeks (cycle 2), n (%) | 21 |
| PFS <6 months | 6 (28.6) |
| PFS ≥6 months | 15 (71.4) |
| at 12 weeks (cycle 4), n (%) | 3 |
| PFS <6 months | 0 (0.0) |
| PFS ≥6 months | 3 (100.0) |

This study aimed to evaluate two main hypotheses. The first goal was to identify whole blood biomarkers capable of predicting ICI treatment response before therapy start. For that, gene expression profiles of both patients with clinical benefit (PFS ≥6 months) and patients without (PFS <6 months) were compared, at baseline. Second goal was to identify blood biomarkers of ICI treatment response, in order to evaluate the efficacy of treatment at an early stage. In this case, a comparison of gene expression was performed between baseline and on-treatment (t=6 weeks) samples, in CB+ as well as in CB− patients, as negative control.

### Sample Collection and Handling

Whole blood samples were collected at two different timepoints during this study. Prior to treatment, at baseline (t=0 weeks), and on treatment, at the end of 2 cycles (t=6 weeks) or 4 cycles (t=12 weeks). These samples were drawn as part of routine clinical care, where a full blood cell count was performed. A PAXgene Blood RNA tube (BD Biosciences, San Jose, CA, USA) was collected for RNA-sequencing and tubes were stored at −80° C. until RNA purification. For 24 of the 29 patients, blood samples were matched between baseline and on-treatment.

### RNA Sequencing

Total RNA was extracted from whole blood using the PAXgene blood miRNA kit (Qiagen, Venlo, Netherlands). RNA samples were treated for globin and ribosomal RNA depletion. Library preparation was performed with the Illumina TruSeq RNA Library Prep Kit v2. Sequencing was performed on Illumina NovaSeq 30 6000 (non-stranded, paired-end 2×150 bp) with an estimated average output of 20-30 million reads/sample.

Raw sequencing data was submitted to quality control (QC) using both FastQC and MultiQC tools (3,4). Mapping and quantification were performed by applying the trimmed paired end reads as input for gene expression analysis using the LITOSeek platform (Novigenix SA, Epalinges, Switzerland). Subsequent reads were aligned, with Hisat2 (6), to the human reference hg38 and using the Salmon tool (7) as reference transcriptome.

### Data Analysis

Acquired data was submitted to normalization, standardization and further evaluated for potential confounding factors and data variability. Initially, metadata was summarized regarding different categories: patient demographics and baseline characteristics; sample handling variables and sample sequencing variables. Descriptive statistics were performed in order to evaluate differences in categorical data repartition. Data was described as number, percentage of categorical data, mean and standard deviation and median for numerical data (such as age). Additionally, prior to differential expression analysis (DEA) all data was studied by unsupervised visualization. In this case, principal components analysis (PCA) was performed applying clinical and technical variables: sex, age, site, sample timepoint, liver metastasis, group (clinical benefit or not), subgroup (BOR), RNA QC variables, sequencing QC analysis, type of treatment and group in each categorical variable. Lastly, output from gene expression analysis was submitted to variance partition analysis for quantification and interpretation of multiple sources of clinical and technical variation.

DEA was performed using standard methods to identify differently expressed genes (DEGs) in each comparison

### Biomarker Selection

Univariate differential expression analyses were complemented by a multivariate feature selection approach, including different machine learning techniques. With the goal of defining final potential biomarkers, the DEGs lists resulting from the different DEA comparisons and the genes selected by multivariate analyses were integrated into a proprietary ranking system and gene selection method (Noviscore). The selected genes were subject to gene enrichment and correlation analyses.

### Functional Analysis

In order to extract information on biological processes from the DEGs, gene enrichment and network analysis were performed. Firstly, data resulting from the blood RNA sequencing (18′000 genes) was processed by gene set enrichment analysis (GSEA). Up and down regulated biological pathways and function were identified by ranking DEGs based on their log 2FC. Upon gene ranking and selection, over representation analysis (ORA) was performed on selected gene sets, using gene function database and the STRINGdb tool for network analysis with link confidence >80% as cutoff parameter.

### Model Training and Performance Estimations

Performance was evaluated by Sparse Partial Least Squares (SPLS) regression and 3-fold cross validation. Survival analysis was performed by Kaplan-Meier curves.

### Result

### Blood Biomarkers Predictive of ICI Treatment Response

### Biomarkers Selection

Differential expression analysis (DEA) between 19 CB+ and 10 CB− patients at baseline was performed and a total of 583 DEGs were identified (pvalue <0.05), of which, 258 were downregulated (−) and 275 were upregulated (+). This list was complemented with genes selected by multivariate analysis, for a final selection of 595 genes (table 1). This list was ranked by the Noviscore system and divided into the top 25 and the top 119 best genes (defined as short list), for performance evaluation (Table 1).

Biological functional analysis of the 595-gene panel identified a 25-gene cluster related to Ribosomal Biogenesis and a 26-gene cluster related to TCR signalling, both upregulated. Moreover it identified 2 downregulated clusters: a 15-gene cluster related to the Interferon signalling and a 4 gene cluster related to cilia motility (Table 1).

Taking into account 7 genes included in the TCR signaling cluster (CD8B, CD8A, CHI3L2, GZMH, IL23, JAKMIP1 and MIAT), CB+ patients exhibit a significant higher expression of this set of genes, in comparison to CB− patients. In parallel, 6 genes (TSR2, GRWD1, RRS1, GLTSCR2, WBSCR22, NOB1) related to ribosomal biogenesis pathway, exhibited a higher expression ratio in CB+ patients versus CB− patients (FIG. 1). These observations validate the discriminatory power of the 2 biological clusters.

### Biomarker Performance

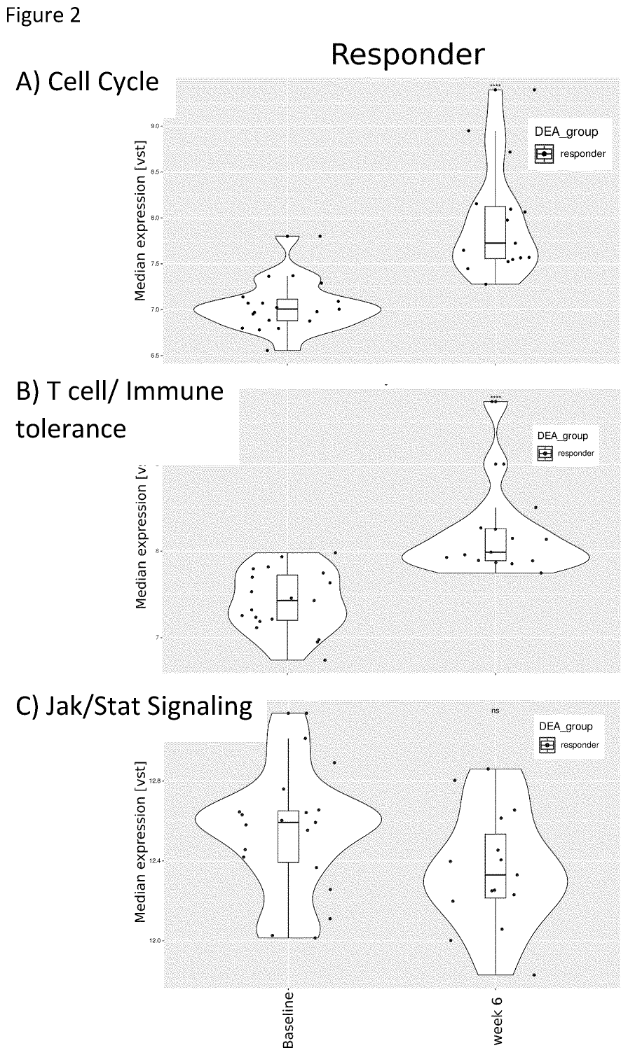

To test the ability of the top ranked 25- and 119-gene lists to predict CB before treatment initiation. we trained predictive models on 29 CB+ and CB− patients at baseline using the top ranked 25- and 119-gene lists as input, and ROC curve were generated. In this training cohort, the 25-gene and the 119-gene models predicted CB from ICI combination therapy with an area under the curve (AUC) of 0.98 and 0.80, respectively, based on cross validation (FIG. 2). Moreover, an initial independent validation of the predictive blood biomarkers, was performed on Lozano et al. 2022 dataset (GSE186144). PBMC from 31 MM patients undergoing anti-PD-1/anti-CTLA-4 combination therapy were analyzed by RNAseq. Response to treatment was evaluated after 6 months. A 64% response rate was observed. The top 25-gene predictive model showed an AUC of 0.68 on their dataset.

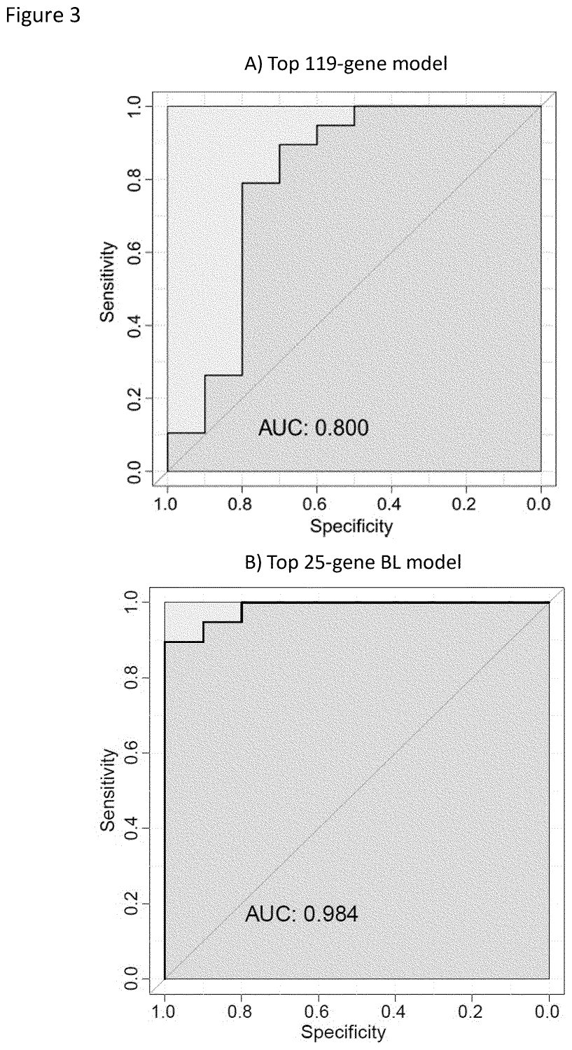

Survival analysis of the 2 patient strata (responder and non-responder) identified by the fitted models on the 119- and 25-gene panels indicates a clear survival difference, demonstrating that the two gene panels are able to predict CB+ and CB− with a highly significant statistical power (p<0.0001) (FIG. 3).

### Blood Biomarkers for Early ICI Response

### Biomarkers Selection

DEA between baseline and wk6 samples of 19 CB+ patients was performed and 364 DEGs were identified (padj<0.05). Of those, 62% (225 genes) were upregulated and 38% (139 genes) were downregulated. This list was complemented with genes selected by multivariate analysis, for a final selection of a 388 genes (Table 2). This list was ranked by the Noviscore system and divided into the top 25 and the top 141 best genes (defined as short list), for performance evaluation (Table 2).

Biological functional analysis of the 388 gene panel from CB+ patients showed a clear enrichment and upregulation of pathways related to T-cell function/immune tolerance (14 genes), Cell Cycle (94 genes) and JAK/STAT signalling (4 genes) was observed (Table 2).

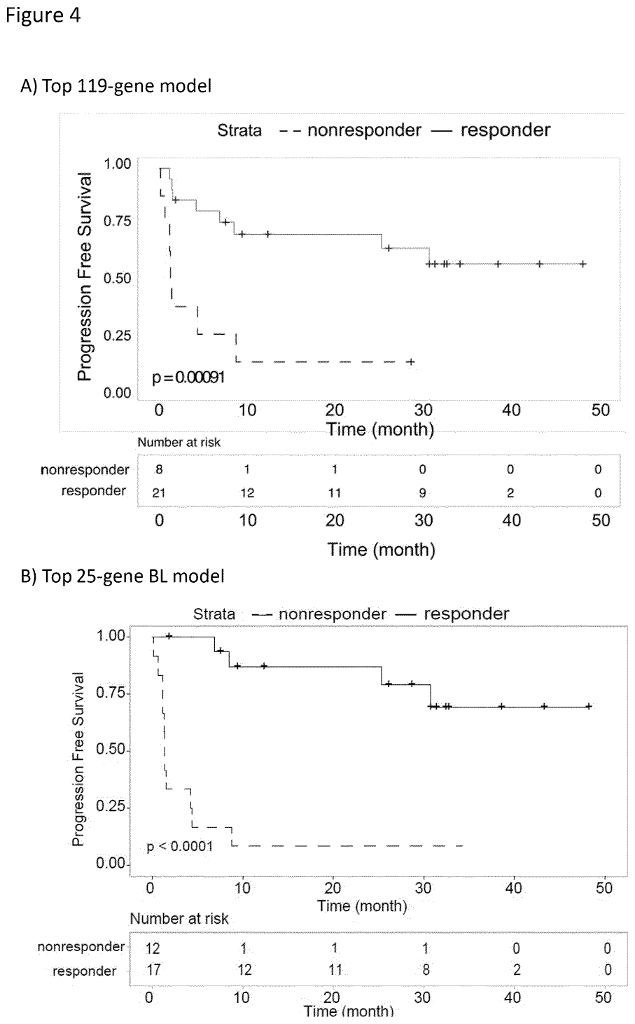

When taking into account 15 genes included in the Cell Cycle cluster (CDCA7, CDC20, PTTG1, CCNB2, RRM2, NCAPG, TYMS, TPX2, MK167, KIF11, CCNA2, EZH2, CCNB1, DLGAP5, GMNN, ASPM, RAD51, TOP2A, BUB1, NCAPH), or 10 genes in the T cell/Immune tolerance regulation genes cluster (PDCD1, IDO1, CTLA4, CCR4, AOC3, LAG3, CXCR3, CD274, CXCL10, CXCL9), the on treatment samples (wk6) exhibit a significant higher expression of these sets of genes, in comparison to baseline. In parallel, the 4 genes related to Jak/Stat Signaling pathway (STAT1, SOCS1, STAT6, TRIM8), exhibited a lower expression in on treatment samples compared to baseline (FIG. 4). These observations validate the discriminatory power of the 2 biological clusters.

### Biomarker Performance

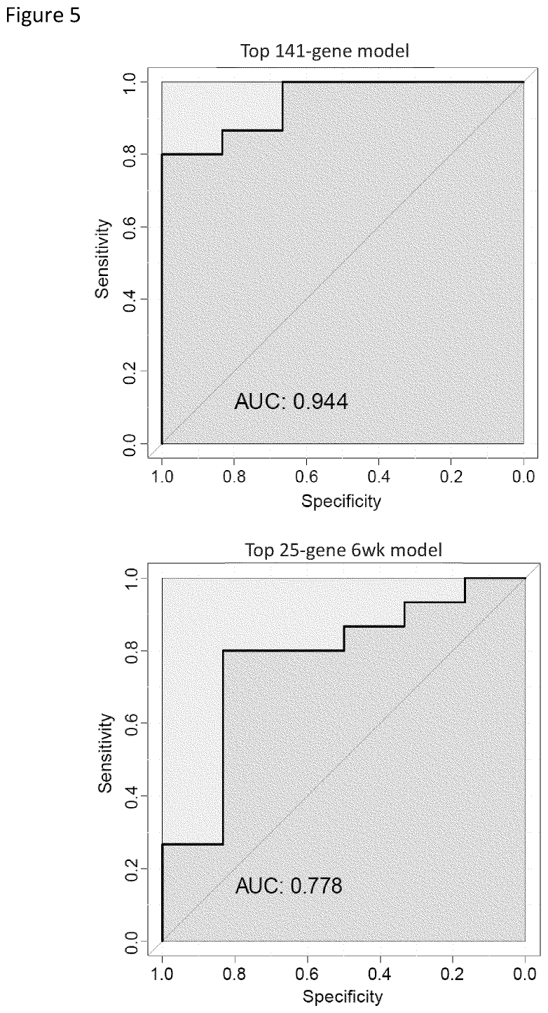

To test the ability of the 141-gene panel as well as of the top ranked 25 genes to identify response to ICI, we trained predictive models on 24 CB+ and CB− patients at 6 wk using the 2 lists as input, and ROC curve were generated. The 25-gene and the 141-gene models demonstrated high value to predict clinical benefit of anti-PD-1/CTLA4 therapy with an area under the curve (AUC) of 0.78 and 0.94, respectively (FIG. 5), based on cross validation.

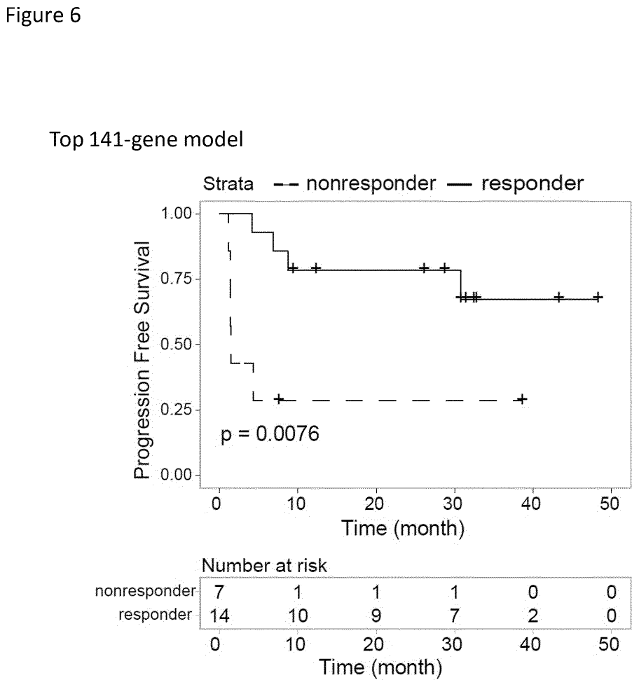

Survival analysis of the 2 patient strata (responder and non-responder) identified by the fitted model on the 141-gene panels (FIG. 6) indicates a clear survival difference (p=0.0076), demonstrating the ability of the biomarkers for identifying patients with clinical benefit early during ICI therapy.

### REFERENCES

1. Postow M A, Chesney J, Pavlick A C, Robert C, Grossman K, McDermott D, et al. Nivolumab and ipilimumab versus ipilimumab in untreated melanoma. New England Journal of Medicine, 2015, 372(21): 2006-2017
2. Garutti, M.; Bonin, S.; Buriolla, S.; Bertoli, E.; Pizzichetta, M. A.; Zalaudek, I.; Puglisi, F. Find the Flame: Predictive Biomarkers for Immunotherapy in Melanoma. Cancers2021, 13,1819.
3. Rotte, A. Combination of CTLA-4 and PD-1 blockers for treatment of cancer. J Exp Clin Cancer Res 38, 255 (2019)
4. Twomey, J. D., Zhang, B. Cancer Immunotherapy Update: FDA-Approved Checkpoint Inhibitors and Companion Diagnostics. AAPS J 23, 39 (2021).
5. Love, MI., Huber, W. & Anders, S. Moderated estimation of fold change and dispersion for RNA-seq data with DESeq2. Genome Biol 15, 550 (2014)
6. Froehler et al., Nucleic Acid Res. 14:5399-5407 (1986)
7. McBride et al., Tetrahedron Lett. 24:246-248 (1983)

1. A method for predicting if a patient having a predetermined disease will respond to a treatment based on immune checkpoint blockade therapy or treatment (ICBT), said method comprising detecting in a biological sample obtained from said patient having a predetermined disease the level of transcription and/or expression and/or activity of a gene panel comprising at least one gene from:
the Ribosomal biogenesis genes cluster,
the TCR signaling genes cluster,
the Cilia genes cluster,
the Interferon pathway genes cluster,
or a combination of one or more thereof,
wherein differential transcription and/or expression and/or activity level of the gene panel, in the biological sample, relative to the level of corresponding transcription and/or expression and/or activity level of the gene panel determined previously, is predictive of the patient's response to said treatment.

2. The method of claim 1, wherein the gene panel comprises at least one gene from those listed in Table 1.

3. The method of claim 1, wherein the gene panel comprises
at least one, at least 2, at least 3, at least 4, at least 5, at least 6, at least 7, at least 8, at least 9, at list 10, at least 11, at least 12, at least 13, at least 14, at least 15, at least 16, at least 17, at least 18, at least 19, at least 20, at least 21, at least 22, at least, 23, at least 24, or at least 25 genes from the best 25″ gene list in Table 1 and/or
at least one, at least 5, at least 8, at least 10, at least 20, at least 30, at least 40, at least 50, at least 60, at least 70, at least 80, at least 90, at least 100, at least 110, or at least 119 genes from the “short” gene list in Table 1.

4. The method of claim 1, wherein the gene panel comprises at least one gene from the TCR signaling genes cluster comprising CD8B, CD8A, CHI3L2, GZMH, IL23, JAKMIP1, or MIAT.

5. The method of claim 1, wherein the gene panel comprises at least one gene from the Ribosomal biogenesis genes cluster comprising TSR2, GRWD1, RRS1, GI-TSCR2, WBSCR22, as NOB1.

6. The method of claim 1, wherein the gene panel comprises at least one gene from the Cilia genes cluster comprising EFCAB2, ENKUR, IQCA1, or IQCD.

7. The method of claim 1, wherein the predetermined disease is cancer.

8. The method of claim 7, wherein the cancer comprises urothelial cancer, urinary bladder cancer, lung cancer, breast cancer, ovarian cancer, cervical cancer, uterus cancer, head and neck cancer, glioblastoma, hepatocellular carcinoma, colon cancer, rectal cancer, kidney cancer, prostate cancer, gastric cancer, bronchus cancer, pancreatic cancer, hepatic cancer, brain cancer, skin cancer, or a combination of one or more thereof.

9. The method of claim 8, wherein the skin cancer comprises metastatic melanoma.

10. The method of claim 1, wherein the treatment based on ICBT comprises a PD-1 inhibitor, a PD-L1 inhibitor, a LAG-3 inhibitor, a TIM-3 inhibitor, a TIGIT inhibitor, a BTLA inhibitor, a CTLA-4 inhibitor, or a combination of one or more thereof.

11. The method of claim 10, wherein the treatment based on ICBT comprises treatment with at least one monoclonal antibody (mAb) specific to PD-1, PD-L1, LAG-3, TIM-3, TIGIT, BTLA, CTLA-4, or a combination of one or more thereof.

12. The method of claim 1, wherein the differential transcription and/or expression and/or activity level of the gene panel corresponds to a differential expression of the transcripts of the gene panel.

13. The method of claim 1, wherein the differential transcription and/or expression and/or activity level of the gene panel corresponds to a downregulated or upregulated expression of the gene panel.

14. The method of claim 13, wherein the downregulated differential transcription and/or expression and/or activity of said one or more genes of the panel corresponds to a decrease equal or superior to about 5% when compared to the level of corresponding transcription and/or expression and/or activity level of the gene panel determined previously.

15. The method of claim 13, wherein the upregulated differential transcription and/or expression and/or activity of said one or more genes of the panel corresponds to an increase equal or superior to about 5% when compared to the level of corresponding transcription and/or expression and/or activity level of the gene panel determined previously.

16. The method of claim 1, wherein, if the patient having a predetermined disease is predicted to respond to said treatment, the treatment based on ICBT is continued or started.

17. The method of claim 1, wherein, if the patient having a predetermined disease is predicted not to respond to said treatment, the method further comprises a step of adapting the treatment based on ICBT.

18. The method of claim 17, wherein the step of adapting the treatment comprises administering a combination therapy, and/or adapting the dose, amount and/or regimen of the treatment based on ICBT.

19. The method of claim 1, wherein the level of corresponding transcription and/or expression and/or activity level of the gene panel determined previously corresponds to a control sample, a reference sample, a group of reference samples, or a reference value.

20. The method of claim 1, wherein the level of transcription and/or expression of the gene panel is performed by whole transcriptome RNA sequencing or targeted RNA seq.

21. The method of claim 1, wherein the level of transcription and/or expression and/or activity of a gene panel is expressed as a score.

22. The method of claim 1, wherein the level of transcription and/or expression and/or activity of a gene panel determined previously is expressed as a score.

23. A method for determining if a patient having a predetermined disease is responsive to a treatment based on immune checkpoint blockade therapy or treatment (ICBT), said method comprising detecting in a biological sample obtained from said patient having a predetermined disease the level of transcription and/or expression and/or activity of a gene panel comprising at least one gene from:
the Cell Cycle genes cluster,
the Jak/Stat Signaling pathway genes cluster,
the T cell/Immune tolerance genes cluster,
or a combination of one or more thereof,
wherein differential transcription and/or expression and/or activity level of the gene panel, in the biological sample, relative to the level of corresponding transcription and/or expression and/or activity level of the gene panel determined previously in a reference sample, is predictive of the patient's response to said treatment.

24. The method of claim 23, wherein the gene panel comprises at least one gene from those listed in Table 2.

25. The method of claim 23, wherein the gene panel comprises at least one gene from the Cell Cycle genes cluster comprising CDCA7, CDC20, PTTG1, CCNB2, RRM2, NCAPG, TYMS, TPX2, MK167, KIF11, CCNA2, EZH2, CCNB1, DLGAP5, GMNN, ASPM, RALD51, TOP2A, BUB1, or NCAPH.

26. The method of claim 23, wherein the gene panel comprises at least one gene from the Jak/Stat Signaling pathway genes cluster comprising STAT1, SOCS1, STAT6, or TRIM8.

27. The method of claim 23, wherein the gene panel comprises at least one gene from the T cell/Immune tolerance genes cluster comprising PDCD1, ID01, CTLA4, CCR4, AOC3, LAG3, CXCR3, CD274, CXCL1, or CXCL9.

28. The method of claim 23, wherein the disease comprises cancer.

29. The method of claim 28, wherein the cancer comprises urothelial cancer, urinary bladder cancer, lung cancer, breast cancer, ovarian cancer, cervical cancer, uterus cancer, head and neck cancer, glioblastoma, hepatocellular carcinoma, colon cancer, rectal cancer, kidney cancer, prostate cancer, gastric cancer, bronchus cancer, pancreatic cancer, hepatic cancer, brain cancer, skin cancer, or a combination of one or more thereof.

30. The method of claim 29, wherein the skin cancer comprises metastatic melanoma.

31. The method of claim 23, wherein the treatment based on ICBT comprises PD-1 inhibitor, a PD-L1 inhibitor, LAG-3 inhibitor, a TIM-3 inhibitor, a TIGIT inhibitor, a BTLA inhibitor, a CTLA-4 inhibitor, or a combination of one or more thereof.

32. The method of claim 23, wherein the treatment based on ICBT comprises treatment with at least one monoclonal antibody (mAb) specific to PD-1, PD-L1, LAG-3, TIM-3, TIGIT, BTLA, CTLA-4, or a combination of one or more thereof.

33. The method of claim 23, wherein the differential transcription and/or expression and/or activity level of the gene panel corresponds to a differential expression of the transcripts of the one or more genes of the panel.

34. The method of claim 23, wherein the differential transcription and/or expression and/or activity level of the gene panel corresponds to a downregulated or upregulated expression of said one or more genes of the panel.

35. The method of claim 34, wherein the downregulated differential transcription and/or expression and/or activity of said one or more genes of the panel corresponds to a decrease equal or superior to about 5% when compared to the level of corresponding transcription and/or expression and/or activity level of the gene panel determined previously.

36. The method of claim 34, wherein the upregulated differential transcription and/or expression and/or activity of said one or more genes of the panel corresponds to an increase equal or superior to about 5% when compared to the level of corresponding transcription and/or expression and/or activity level of the gene panel determined previously.

37. The method of claim 23, wherein, if the patient having a predetermined disease is determined as responsive to said treatment, the treatment is continued.

38. The method of claim 23, wherein, if the patient having a predetermined disease is determined as non-responsive to said treatment, the method further comprises a step of adapting the treatment.

39. The method of claim 38, wherein the step of adapting the treatment comprises changing the treatment for another treatment or administering a combination therapy, and/or adapting the dose and/or regimen of the treatment based on ICBT.

40. The method of claim 23, wherein the level of transcription and/or expression of a gene panel is performed by whole transcriptome RNA sequencing or targeted RNA seq.

41. The method of claim 23, wherein the level of transcription and/or expression and/or activity of the gene panel is detected in the biological sample about 1 to about 16 weeks, about 2 to about 14 weeks, about 2 to about 12 weeks, or about 2 to about 10 weeks, after the treatment based on ICBT has started.

42. A computer-implemented method for implementing a method for predicting if a patient having a predetermined disease will respond to a treatment based on immune checkpoint blockade therapy or treatment (ICBT) of claim 1, said computer-implemented method comprising:
i) scoring the level of transcription and/or expression and/or activity of the gene panel in the biological sample of the patient,
ii) comparing the determined score to the score of the gene panel determined previously, whereby difference in the score, in the biological sample, relative to the score of the gene panel determined previously, is predictive of the patient's response to said treatment.

43. A computer-implemented method for implementing a method for determining if a patient having a predetermined disease is responsive to a treatment based on immune checkpoint blockade therapy or treatment (ICBT) of claim 23, said computer-implemented method comprising:
i) scoring the level of transcription and/or expression and/or activity of the gene panel in the biological sample of the patient,
ii) comparing the determined score of the gene panel determined previously, whereby difference in the score, in the biological sample, relative to the score of the gene panel determined previously, is indicative of whether the patient is responsive or not to said treatment.

44. (canceled)

45. (canceled)

46. A kit for performing a method according to claim 1, said kit comprising
a) means for determining the level of transcription and/or expression and/or activity of said gene panel in a biological sample from said patient, and
b) instructions for use.

47.-49. (canceled)
